(19) United States
(12) Patent Application Publication
(10) Pub. No.: US 2025/0064809 A1
(43) Pub. Date: Feb. 27, 2025

(54) Inhibitors of Mutant RET Kinases for use in Treating Cancer

(71) Applicant: Stemline Therapeutics, Inc., New York, NY (US)

(72) Inventor: Enrique Poradosu, Brookline, MA (US)

(73) Assignee: Stemline Therapeutics, Inc., New York, NY (US)

(21) Appl. No.: 18/626,722

(22) Filed: Apr. 4, 2024

Related U.S. Application Data
(63) Continuation of application No. PCT/US2022/078023, filed on Oct. 13, 2022.
(60) Provisional application No. 63/358,897, filed on Jul. 7, 2022.
(60) Provisional application No. 63/256,037, filed on Oct. 15, 2021.

(51) Int. Cl. A61K 31/519

(52) U.S. Cl. CPC A61K 31/519

(57) ABSTRACT

Disclosed herein are methods and pharmaceutical compositions for treating a cancer associated with RET kinase activity in a subject in need thereof, wherein the cancer has developed resistance to prior therapy and/or wherein the subject has a solvent front mutation in the RET kinase.

### CROSS-REFERENCE TO RELATED APPLICATIONS

This application claims priority to U.S. Provisional Application No. 63/256,037, filed on Oct. 15, 2021, and U.S. Provisional Application No. 63/358,897, filed on Jul. 7, 2022, the disclosure of each of which is hereby incorporated by reference in its entirety.

### BACKGROUND OF THE DISCLOSURE

Rearranged during transfection (RET) kinase is a transmembrane receptor protein kinase that has been implicated in certain cancers. Complex formation of glycosylphosphatidylinositol (GPI)-anchored co-receptors and glial derived neurotrophic factor (GDNF) family ligands induces the dimerization of two RET receptors and triggers trans-phosphorylation of specific RET tyrosine residues, which in turn activates signal transduction pathways that regulate cell survival, differentiation, proliferation, migration, and chemotaxis.

Activating point mutations in RET kinase have been associated with medullary thyroid cancer (MTC), and RET fusions with non-small cell lung cancer (NSCLC) and papillary thyroid carcinoma (PTC) (Liu, X. et al., Cancer Drug Resist. 2020, 3, 472-481). The oncogenic association of RET has prompted the development of RET-selective tyrosine kinase inhibitors (TKI) such as selpercatinib and pralsetinib. Although these US Food and Drug Administration (FDA)-approved inhibitors exhibit effective antitumor activities, the inhibitors are prone to resistance by various RET mutations.

Recent X-ray crystallographic studies have provided insight into the structural basis of resistance. In one study, the multikinase inhibitor nintedanib was used to gain an understanding of the interactions between TKIs and RET kinase mutations (Terzyan, S. S. et al., J. Biol. Chem. 2019, 294(27), 10428-10437). The crystal structures of a RET-nintedanib complex and a mutant RET kinase (G810A) revealed that mutations at specific amino acid residues of RET that alter the hydrophobic interaction with nintedanib are closely associated with the development of resistance. In another study, crystallographic structures of RET kinase with the selective inhibitors selpercatinib or pralsetinib showed that although these two drugs avoid resistance induced by gatekeeper mutations by adopting an unconventional binding mode to RET, both drugs are vulnerable to other mutations such as solvent front mutations of RET kinase (Subbiah, V. et al., Ann. Oncol. 2021, 32(2), 261-268).

RET G810 solvent front mutations (G810R, G810S, G810C, and G810V) have been linked with acquired resistance to RET inhibition with selpercatinib (Solomon, B. J. et al. J. Thorac. Oncol. 2020, 15(4), 541-549). RET solvent front mutations are also driven by off-target, RET-independent mechanisms, such as MET or KRAS amplification, in studies that investigated specimen from selpercatinib- and pralsetinib-resistant patients (Lin J. J. et al. Ann. Oncol. 2020, 31(12), 1725-1733).

Accordingly, there is a need for inhibitors of RET kinase that are active against both gatekeeper mutations and solvent front mutations.

### SUMMARY OF THE DISCLOSURE

Described herein, in certain embodiments, are methods for treating a cancer associated with RET kinase activity in a subject in need thereof, comprising administering to the subject an effective amount of a compound of Formula (I), or a pharmaceutically acceptable salt thereof, wherein the cancer has developed resistance following prior therapy and/or wherein the subject has a solvent front mutation in the RET kinase. Also provided herein are methods of inhibiting a mutant RET kinase, comprising contacting the mutant RET kinase with an effective amount of a compound of Formula (I), or a pharmaceutically acceptable salt thereof.

The following embodiments are encompassed.

Embodiment 1 is a method for treating a cancer associated with RET kinase activity in a subject in need thereof, comprising administering to the subject an effective amount of a compound of Formula (I), or a pharmaceutically acceptable salt thereof, wherein the cancer has developed resistance following prior therapy and/or wherein the subject has a solvent front mutation in the RET kinase, and wherein the compound of Formula (I) has the following structure:

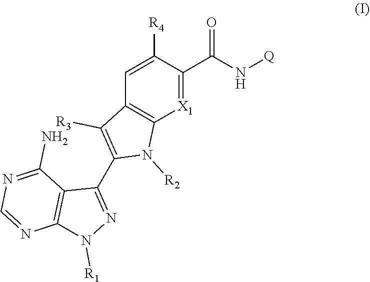

wherein:
X₁ is N or CH;
R₁ is C₁-C₆ alkyl or C₃-C₆ cycloalkyl, each of which is optionally substituted by 1-5 substituents independently selected from halo, hydroxy, and —O(C₁-C₆ alkyl);
R₂ is H or C₁-C₆ alkyl;
R₃ is halo or C₁-C₆ alkyl;
R₄ is H or halo; and
Q is C₁-C₆ alkyl or C₃-C₆ cycloalkyl, each of which is optionally substituted by 1-5 substituents independently selected from hydroxy, —O(C₁-C₆ alkyl), and halo.

Embodiment 2 is the method of embodiment 1, wherein:
X₁ is CH;
R₁ is C₃-C₆ alkyl optionally substituted by 1-3 substituents independently selected from halo, hydroxy, and —O(C₁-C₆ alkyl);
R₂ is H;
R₃ is halo;
R₄ is H; and
Q is C₁-C₃ alkyl or C₃-C₆ cycloalkyl, each of which is optionally substituted by 1-3 substituents independently selected from hydroxy and —O(C₁-C₃ alkyl).

Embodiment 3 is the method of embodiment 2, wherein:
R₁ is isopropyl or tert-butyl;
R₃ is Cl; and
Q is —CH₃, —CH₂CH₂OH, —(CH₂)₃OCH(CH₃)₂, or cyclopropyl.

Embodiment 4 is the method of any one of embodiments 1-3, wherein the compound of Formula (I) is:

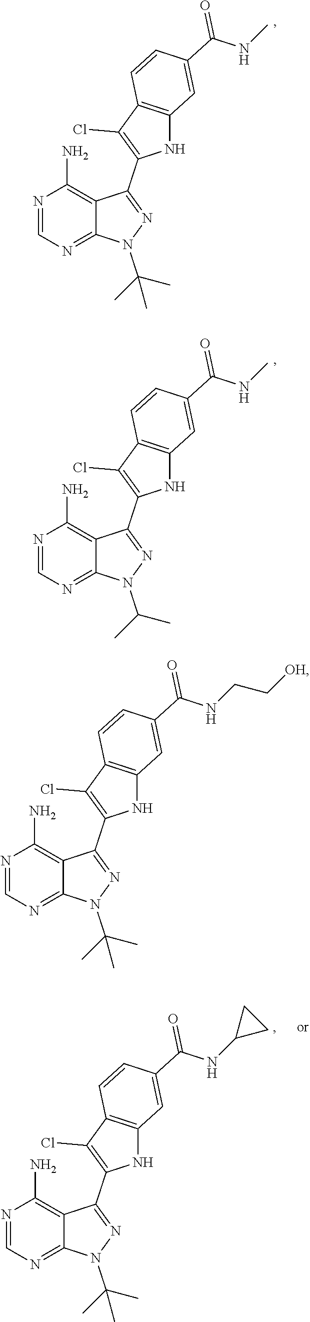

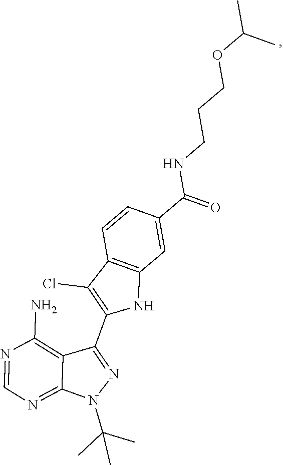 [SMILES: CC(C)OCCCNC(=O)c1ccc2c(Cl)c(-c3nn(C(C)(C)C)c4ncnc(N)c34)[nH]c2c1]

or a pharmaceutically acceptable salt thereof.

Embodiment 5 is the method of any one of embodiments 1-4, wherein the compound of Formula (I) is:

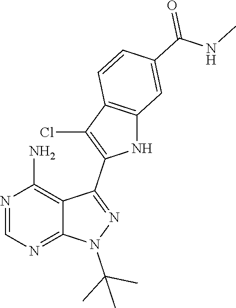 [SMILES: CNC(=O)c1ccc2c(Cl)c(-c3nn(C(C)(C)C)c4ncnc(N)c34)[nH]c2c1]

or a pharmaceutically acceptable salt thereof.

Embodiment 6 is the method of any one of embodiments 1-4, wherein the compound of Formula (I) is:

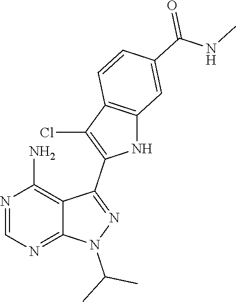 [SMILES: CNC(=O)c1ccc2c(Cl)c(-c3nn(C(C)C)c4ncnc(N)c34)[nH]c2c1]

or a pharmaceutically acceptable salt thereof.

Embodiment 7 is the method of any one of embodiments 1-4, wherein the compound of Formula (I) is:

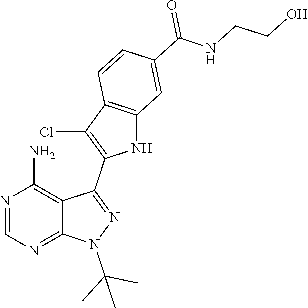 [SMILES: CC(C)(C)n1nc(-c2[nH]c3cc(C(=O)NCCO)ccc3c2Cl)c2c(N)ncnc21]

or a pharmaceutically acceptable salt thereof.

Embodiment 8 is the method of any one of embodiments 1-4, wherein the compound of Formula (I) is:

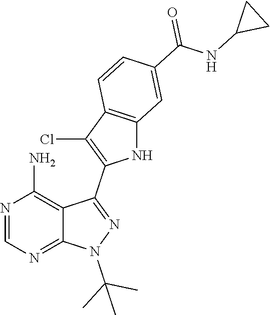 [SMILES: CC(C)(C)n1nc(-c2[nH]c3cc(C(=O)NC4CC4)ccc3c2Cl)c2c(N)ncnc21]

or a pharmaceutically acceptable salt thereof.

Embodiment 9 is the method of any one of embodiments 1-4, wherein the compound of Formula (I) is:

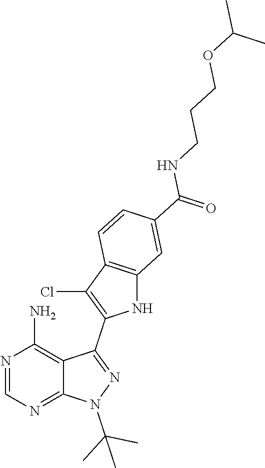 [SMILES: CC(C)OCCCNC(=O)c1ccc2c(Cl)c(-c3nn(C(C)(C)C)c4ncnc(N)c34)[nH]c2c1]

or a pharmaceutically acceptable salt thereof.

Embodiment 10 is the method of any one of embodiments 1-9, wherein the subject previously responded to the prior therapy.

Embodiment 11 is the method of any one of embodiments 1-10, wherein the subject no longer responds to the prior therapy.

Embodiment 12 is the method of any one of embodiments 1-11, wherein the prior therapy is a kinase inhibitor, immunotherapy, chemotherapy, surgery, or radiation.

Embodiment 13 is the method of embodiment 12, wherein the prior therapy is a kinase inhibitor.

Embodiment 14 is the method of embodiment 13, wherein the kinase inhibitor is a selective RET kinase inhibitor or a multikinase inhibitor.

Embodiment 15 is the method of embodiment 14, wherein the selective RET kinase inhibitor is selpercatinib or pralsetinib.

Embodiment 16 is the method of embodiment 14, wherein the multikinase inhibitor is nintedanib, vandetanib, cabozantinib, lenvatinib, RXDX-105, sunitinib, sorafenib, alectinib, ponatinib, or regorafenib.

Embodiment 17 is the method of any one of embodiments 1-16, wherein the cancer is a malignant neoplasm, a malignant tumor, or a solid tumor.

Embodiment 18 is the method of any one of embodiments 1-17, wherein the cancer is leukemia, lung cancer, colon cancer, breast cancer, ovarian cancer, prostate cancer, liver cancer, pancreatic cancer, brain cancer, skin cancer, thyroid cancer, salivary gland cancer, endocrine cancer, urothelial cancer, uterine cancer, fallopian tube cancer, gastrointestinal cancer, or esophageal cancer.

Embodiment 19 is the method of embodiment 18, wherein the cancer is medullary thyroid cancer, non-small cell lung cancer, lung carcinosarcoma, lung adenocarcinoma, atypical lung carcinoid, multiple endocrine neoplasia type 2, ovarian epithelial carcinoma, uterine carcinosarcoma, fallopian tube adenocarcinoma, chronic myelomonocytic leukemia (CMML), melanoma, basal cell carcinoma, Merkel cell carcinoma, salivary gland adenocarcinoma, papillary thyroid carcinoma (PTC), anaplastic thyroid carcinoma, meningioma, esophageal adenocarcinoma, gastric adenocarcinoma, ureter urothelial carcinoma, duodenal adenocarcinoma, or colorectal adenocarcinoma.

Embodiment 20 is the method of any one of embodiments 1-19, wherein the resistance of the cancer is due to a solvent front mutation in the RET kinase.

Embodiment 21 is the method of any one of embodiments 1-20, wherein the solvent front mutation is at G810 in the amino acid sequence of the RET kinase.

Embodiment 22 is the method of any one of embodiments 1-21, wherein the solvent front mutation is G810A, G810C, G810R, G810V, or G810S.

Embodiment 23 is the method of any one of embodiments 1-22, wherein the RET kinase further comprises a RET fusion translocation and/or a mutation at the RET gatekeeper residue V804.

Embodiment 24 is the method of embodiment 23, wherein the RET fusion translocation is KIF5B-RET or CCDC6-RET.

Embodiment 25 is the method of embodiment 23 or 24, wherein the RET gatekeeper residue V804 is RET^{V804M}.

Embodiment 26 is the method of any one of embodiments 1-25, wherein the solvent front mutation, the RET fusion translocation, and/or the mutation at the RET gatekeeper residue V804 in the RET kinase is identified by a detection method comprising evaluating circulating tumor (cell-free) DNA and/or evaluating a tissue biopsy.

Embodiment 27 is the method of embodiment 26, wherein the detection method comprises sequencing.

Embodiment 28 is a method of inhibiting a mutant RET kinase, comprising contacting the mutant RET kinase with an effective amount of a compound of Formula (I):

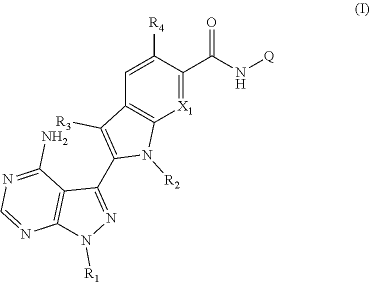

or a pharmaceutically acceptable salt thereof, wherein:
X₁ is N or CH;
R₁ is C₁-C₆ alkyl or C₃-C₆ cycloalkyl, each of which is optionally substituted by 1-5 substituents independently selected from halo, hydroxy, and —O(C₁-C₆ alkyl);
R₂ is H or C₁-C₆ alkyl;
R₃ is halo or C₁-C₆ alkyl;
R₄ is H or halo; and
Q is C₁-C₆ alkyl or C₃-C₆ cycloalkyl, each of which is optionally substituted by 1-5 substituents independently selected from hydroxy, —O(C₁-C₆ alkyl), and halo.

Embodiment 29 is the method of embodiment 28, wherein:
X₁ is CH;
R₁ is C₃-C₆ alkyl optionally substituted by 1-3 substituents independently selected from halo, hydroxy, and —O(C₁-C₆ alkyl);
R₂ is H;
R₃ is halo;
R₄ is H; and
Q is C₁-C₃ alkyl or C₃-C₆ cycloalkyl, each of which is optionally substituted by 1-3 substituents independently selected from hydroxy and —O(C₁-C₃ alkyl).

Embodiment 30 is the method of embodiment 29, wherein:
R₁ is isopropyl or tert-butyl;
R₃ is Cl; and
Q is —CH₃, —CH₂CH₂OH, —(CH₂)₃OCH(CH₃)₂, or cyclopropyl.

Embodiment 31 is the method of any one of embodiments 28-30, wherein the compound of Formula (I) is:

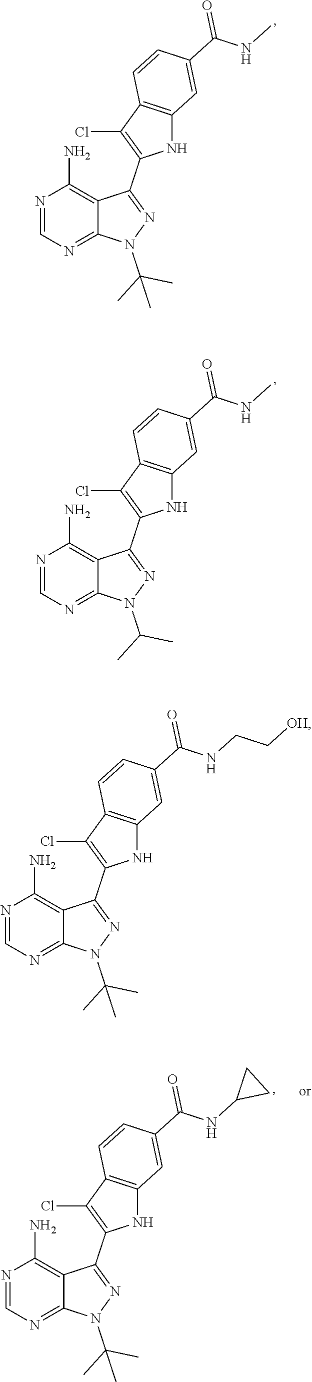 [SMILES: CNC(=O)c1ccc2c(Cl)c(-c3nn(C(C)(C)C)c4ncnc(N)c34)[nH]c2c1 ; CNC(=O)c1ccc2c(Cl)c(-c3nn(C(C)C)c4ncnc(N)c34)[nH]c2c1 ; CC(C)(C)n1nc(-c2[nH]c3cc(C(=O)NCCO)ccc3c2Cl)c2c(N)ncnc21 ; CC(C)(C)n1nc(-c2[nH]c3cc(C(=O)NC4CC4)ccc3c2Cl)c2c(N)ncnc21]

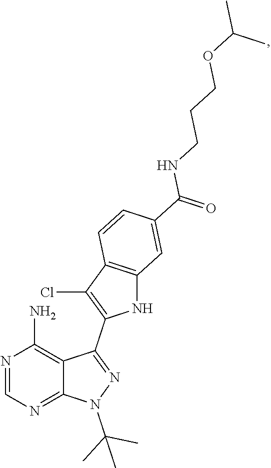 [SMILES: CC(C)OCCCNC(=O)c1ccc2c(Cl)c(-c3nn(C(C)(C)C)c4ncnc(N)c34)[nH]c2c1]

or a pharmaceutically acceptable salt thereof.

Embodiment 32 is the method of any one of embodiments 28-31, wherein the compound of Formula (I) is:

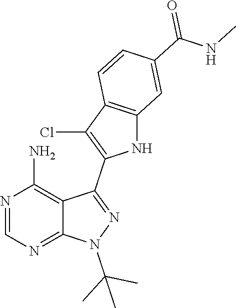 [SMILES: CNC(=O)c1ccc2c(Cl)c(-c3nn(C(C)(C)C)c4ncnc(N)c34)[nH]c2c1]

or a pharmaceutically acceptable salt thereof.

Embodiment 33 is the method of any one of embodiments 28-31, wherein the compound of Formula (I) is:

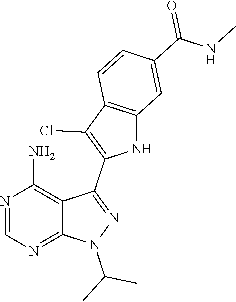 [SMILES: CNC(=O)c1ccc2c(Cl)c(-c3nn(C(C)C)c4ncnc(N)c34)[nH]c2c1]

or a pharmaceutically acceptable salt thereof.

Embodiment 34 is the method of any one of embodiments 28-31, wherein the compound of Formula (I) is:

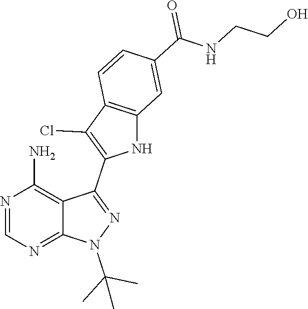 [SMILES: CC(C)(C)n1nc(-c2[nH]c3cc(C(=O)NCCO)ccc3c2Cl)c2c(N)ncnc21]

or a pharmaceutically acceptable salt thereof.

Embodiment 35 is the method of any one of embodiments 28-31, wherein the compound of Formula (I) is:

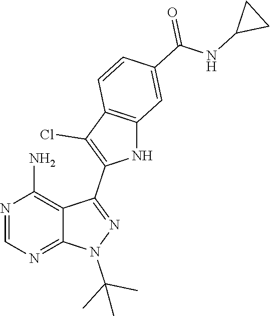 [SMILES: CC(C)(C)n1nc(-c2[nH]c3cc(C(=O)NC4CC4)ccc3c2Cl)c2c(N)ncnc21]

or a pharmaceutically acceptable salt thereof.

Embodiment 36 is the method of any one of embodiments 28-31, wherein the compound of Formula (I) is:

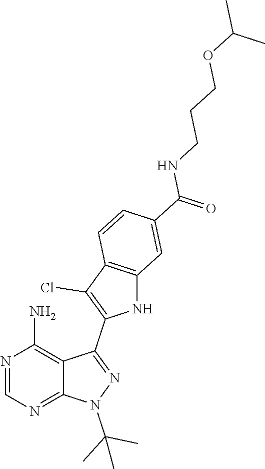 [SMILES: CC(C)OCCCNC(=O)c1ccc2c(Cl)c(-c3nn(C(C)(C)C)c4ncnc(N)c34)[nH]c2c1]

or a pharmaceutically acceptable salt thereof.

Embodiment 37 is the method of any one of embodiments 28-36, wherein the method is an in vitro method.

Embodiment 38 is the method of any one of embodiments 28-36, wherein the method is an in vivo method.

Embodiment 39 is the method of any one of embodiments 28-38, wherein the mutant RET kinase is due to resistance developed from a prior therapy.

Embodiment 40 is the method of embodiment 39, wherein the prior therapy is a kinase inhibitor, immunotherapy, chemotherapy, surgery, or radiation.

Embodiment 41 is the method of embodiment 40, wherein the prior therapy is a kinase inhibitor.

Embodiment 42 is the method of embodiment 41, wherein the kinase inhibitor is a selective RET kinase inhibitor or a multikinase inhibitor.

Embodiment 43 is the method of embodiment 42, wherein the selective RET kinase inhibitor is selpercatinib or pralseltinib.

Embodiment 44 is the method of embodiment 42, wherein the multikinase inhibitor is nintedanib, vandetanib, cabozantinib, lenvatinib, RXDX-105, sunitinib, sorafenib, alectinib, ponatinib, or regorafenib.

Embodiment 45 is the method of any one of embodiments 28-44, wherein the mutant RET kinase comprises a solvent front mutation.

Embodiment 46 is the method of embodiment 45, wherein the solvent front mutation is at G810 in the amino acid sequence of the RET kinase.

Embodiment 47 is the method of embodiment 46, wherein the solvent front mutation is G810A, G810C, G810R, G810V, or G810S.

Embodiment 48 is the method of any one of embodiments 28-47, wherein the RET kinase further comprises a RET fusion translocation and/or a mutation at the RET gatekeeper residue V804.

Embodiment 49 is the method of embodiment 48, wherein the RET fusion translocation is KIF5B-RET or CCDC6-RET.

Embodiment 50 is the method of embodiment 48 or 49, wherein the RET gatekeeper residue V804 is RET^{V804M}.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows % inhibition curves of Compound A against RET WT and mutant RET kinases.

FIG. 1B shows % inhibition curves of selpercatinib against RET WT and mutant RET kinases.

FIG. 1C shows % inhibition curves of pralsetinib against RET WT and mutant RET kinases.

FIG. 1D shows % inhibition curves of staurosporine against RET WT and mutant RET kinases.

FIG. 2 shows the fold changes in IC₅₀ values for mutant RET kinases treated with Compound A, selpercatinib, pralsetinib, or staurosporine. Data are normalized to IC₅₀ values for RET WT.

FIG. 3A shows % of surviving cells of a Ba/F3 cell line stably expressing kinesin family 5B (KIF5B)-RET with a wildtype RET domain (Ba/F3 KIF5B-RET) treated with Compound A, pralsetinib, or selpercatinib.

FIG. 3B shows % of surviving cells of a Ba/F3 cell line stably expressing KIF5B-RET with a G810R mutant RET domain (Ba/F3 KIF5B-RET-G810R) treated with Compound A, pralsetinib, or selpercatinib.

FIG. 3C shows % of surviving cells of a Ba/F3 cell line stably expressing KIF5B-RET with a G810C mutant RET domain (Ba/F3 KIF5B-RET-G810C) treated with Compound A, pralsetinib, or selpercatinib.

FIG. 3D shows % of surviving cells of a Ba/F3 cell line stably expressing KIF5B-RET with a G810S mutant RET domain (Ba/F3 KIF5B-RET-G810S) treated with Compound A, pralsetinib, or selpercatinib.

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless the context requires otherwise. Furthermore, use of the term “including” as well as other forms, such as “include”, “includes,” and “included,” is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

“Solvent front mutation” or “solvent front mutations” refers to one or more amino acid mutations located at the solvent front of the protein (i.e., at a position on the protein that is exposed to solvent). In some embodiments, a solvent front mutation causes direct steric hindrance to inhibitor binding and/or destabilizes the favorable electrostatic interactions between the inhibitor and its binding site.

“Alkyl” refers to an unbranched or branched saturated hydrocarbon chain. As used herein, alkyl has 1 to 20 carbon atoms (i.e., C₁-C₂₀ alkyl), 1 to 10 carbon atoms (i.e., C₁-C₁₀ alkyl), 1 to 6 carbon atoms (i.e., C₁-C₆ alkyl) or 1 to 3 carbon atoms (i.e., C₁-C₃ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), isobutyl (i.e., —CH₂CH(CH₃)₂), sec-butyl (i.e., —CH(CH₃)CH₂CH₃), and tert-butyl (i.e., —C(CH₃)₃); and “propyl” includes n-propyl (i.e., —(CH₂)₂CH₃) and isopropyl (i.e., —CH(CH₃)₂).

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond). As used herein, cycloalkyl has from 3 to 20 ring carbon atoms (i.e., C₃-C₂₀ cycloalkyl), 3 to 10 ring carbon atoms (i.e., C₃-C₁₀ cycloalkyl), or 3 to 6 ring carbon atoms (i.e., C₃-C₆ cycloalkyl). Cycloalkyl also includes “spiro cycloalkyl” when there are two positions for substitution on the same carbon atom. Non-limiting examples of cycloalkyl groups include, for example, cyclopropyl, cyclobutyl, cyclopentyl, cyclohexyl, cycloheptyl, and cyclooctyl. Further, the term cycloalkyl is intended to encompass any non-aromatic ring which may be fused to an aryl ring, regardless of the attachment to the remainder of the molecule.

“Halogen” or “halo” includes fluoro, chloro, bromo, and iodo.

“Hydroxy” refers to an —OH group.

The terms “optional” or “optionally” means that the subsequently described event or circumstance may or may not occur and that the description includes instances where said event or circumstance occurs and instances in which it does not. Also, the term “optionally substituted” refers to any one or more hydrogen atoms on the designated atom or group may or may not be replaced by a moiety other than hydrogen.

Any compound or formula described herein is intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S ¹⁸F, ³⁶Cl, ¹²³I and ¹²⁵I, respectively. Various isotopically labeled compounds of the present disclosure, for example those into which radioactive isotopes such as ³H, ¹³C and ¹⁴C are incorporated, are included in this disclosure. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds described herein in which from 1 to n hydrogens attached to a carbon atom is/are replaced by deuterium, in which n is the number of hydrogens in the molecule. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

“Pharmaceutically acceptable” refers to compounds, salts, compositions, dosage forms, and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines. Specific examples of suitable amines include, by way of example only, isopropylamine, trimethyl amine, diethyl amine, tri(isopropyl)amine, tri(n-propyl)amine, ethanolamine, 2-dimethylaminoethanol, piperazine, piperidine, morpholine, N-ethylpiperidine and the like.

The compounds disclosed herein, or their pharmaceutically acceptable salts, may include an asymmetric center and may thus give rise to enantiomers, diastereomers, and other stereoisomeric forms that may be defined, in terms of absolute stereochemistry, as (R)- or (S)- or, as (D)- or (L)- for amino acids. The disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

“Tautomer” refers to alternate forms of a compound that differ in the position of a proton, such as enol-keto and imine-enamine tautomers, or the tautomeric forms of heteroaryl groups containing a ring atom attached to both a ring —NH-moiety and a ring ═N moiety such as pyrazoles, imidazoles, benzimidazoles, triazoles, and tetrazoles. All tautomeric forms of the compounds described herein are intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

“Diastereoisomers” are stereoisomers that have at least two asymmetric atoms, but which are not mirror-images of each other.

As used herein, “pharmaceutically acceptable carrier” or “pharmaceutically acceptable excipient” or “excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Effective amount” or dose of a compound or a composition refers to that amount of the compound or the composition that results in an intended result as desired based on the disclosure herein. Effective amounts can be determined by standard pharmaceutical procedures in cell cultures or experimental animals including, without limitation, by determining the LD₅₀ (the dose lethal to 50% of the population) and the ED₅₀ (the dose therapeutically effective in 50% of the population).

“Therapeutically effective amount” or dose of a compound or a composition refers to that amount of the compound or the composition that results in reduction or inhibition of symptoms or a prolongation of survival in a subject (i.e., a human patient). The results may require multiple doses of the compound or the composition.

“Treating” or “treatment” of a disease in a subject refers to 1) preventing the disease from occurring in a patient that is predisposed or does not yet display symptoms of the disease; 2) inhibiting the disease or arresting its development; or 3) ameliorating or causing regression of the disease. As used herein, “treatment” or “treating” is an approach for obtaining beneficial or desired results including clinical results. For the purposes of this disclosures, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a subject. Also encompassed by “treatment” is a reduction of pathological consequence of the disease or disorder. The methods of the invention contemplate any one or more of these aspects of treatment.

As used herein, the terms “subject(s)” and “patient(s)” mean any mammal. In some embodiments, the mammal is a human. In some embodiments, the mammal is a non-human, such as a primate, dog, cat, rabbit, or rodent. None of the terms require or are limited to situations characterized by the supervision (e.g., constant or intermittent) of a health care worker (e.g., a doctor, a registered nurse, a nurse practitioner, a physician's assistant, an orderly or a hospice worker).

As used herein, the terms “pharmaceutical composition” or “medicament” refer to a composition suitable for pharmaceutical use in a subject, e.g., as a RET kinase inhibitor.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

The disclosure provides compounds as inhibitors of RET kinase having the structure of Formula (I):

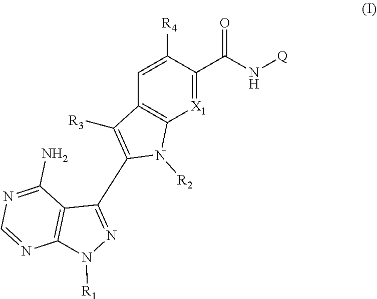

or a pharmaceutically acceptable salt thereof, wherein:
X₁ is N or CH;
R₁ is C₁-C₆ alkyl or C₃-C₆ cycloalkyl, each of which is optionally substituted by 1-5 substituents independently selected from halo, hydroxy, and —O(C₁-C₆ alkyl);
R₂ is H or C₁-C₆ alkyl;
R₃ is halo or C₁-C₆ alkyl;
R₄ is H or halo;
Q is C₁-C₆ alkyl or C₃-C₆ cycloalkyl, each of which is optionally substituted by 1-5 substituents independently selected from hydroxy, —O(C₁-C₆ alkyl), and halo.

In some embodiments, X₁ is N. In some embodiments, X₁ is CH.

In some embodiments, R₁ is C₁-C₆ alkyl optionally substituted by 1-5 substituents independently selected from hydroxy, —O(C₁-C₆ alkyl), and halo. In some embodiments, R₁ is C₁-C₃ alkyl optionally substituted by 1-5 substituents independently selected from halo, hydroxy, and
—O(C₁-C₆ alkyl). In some embodiments, R₁ is C₁-C₃ alkyl optionally substituted by 1-3 substituents independently selected from halo, hydroxy, and —O(C₁-C₆ alkyl). In some embodiments, R₁ is C₁ alkyl (i.e., methyl) optionally substituted by 1-5 substituents independently selected from halo, hydroxy, and —O(C₁-C₆ alkyl). In some embodiments, R₁ is C₂ alkyl (i.e., ethyl) optionally substituted by 1-5 substituents independently selected from halo, hydroxy, and —O(C₁-C₆ alkyl). In some embodiments, R₁ is C₃ alkyl, such as n-propyl or isopropyl, optionally substituted by 1-5 substituents independently selected from halo, hydroxy, and —O(C₁-C₆ alkyl). In some embodiments, R₁ is C₄ alkyl, such as n-butyl, sec-butyl, or tert-butyl, optionally substituted by 1-5 substituents independently selected from halo, hydroxy, and —O(C₁-C₆ alkyl). In some embodiments, R₁ is C₅ alkyl, such as n-pentyl, sec-pentyl, or iso-pentyl, optionally substituted by 1-5 substituents independently selected from halo, hydroxy, and —O(C₁-C₆ alkyl). In some embodiments, R₁ is C₆ alkyl, such as n-hexyl, optionally substituted by 1-5 substituents independently selected from halo, hydroxy, and —O(C₁-C₆ alkyl). In some embodiments, R₁ is C₁-C₆ alkyl optionally substituted by 1-5 substituents independently selected from hydroxy, —O(C₁-C₃ alkyl) such as methoxy or ethoxy, and halo (such as F, Cl, or Br).

In some embodiments, R₁ is C₃-C₆ cycloalkyl optionally substituted by 1-5 substituents independently selected from hydroxy, —O(C₁-C₆ alkyl), and halo. In some embodiments, R₁ is C₃-C₆ cycloalkyl optionally substituted by 1-3 substituents independently selected from hydroxy, —O(C₁-C₆ alkyl), and halo. In some embodiments, R₁ is C₃ cycloalkyl (i.e., cyclopropyl) optionally substituted by 1-5 substituents independently selected from hydroxy, —O(C₁-C₆ alkyl), and halo. In some embodiments, R₁ is C₄ cycloalkyl (i.e., cyclobutyl) optionally substituted by 1-5 substituents independently selected from hydroxy, —O(C₁-C₆ alkyl), and halo. In some embodiments, R₁ is C₅ cycloalkyl (i.e., cyclopentyl) optionally substituted by 1-5 substituents independently selected from hydroxy, —O(C₁-C₆ alkyl), and halo. In some embodiments, R₁ is C₆ cycloalkyl (i.e., cyclohexyl) optionally substituted by 1-5 substituents independently selected from hydroxy, —O(C₁-C₆ alkyl), and halo. In some embodiments, R₁ is C₃-C₆ cycloalkyl optionally substituted by 1-5 substituents independently selected from hydroxy, —O(C₃-C₆ alkyl) such as methoxy or ethyoxy, and halo (such as F, Cl, or Br).

In some embodiments, R₂ is H. In some embodiments, R₂ is C₁-C₆ alkyl. In some embodiments, R₂ is C₁ alkyl (i.e., methyl). In some embodiments, R₂ is C₂ alkyl (i.e., ethyl). In some embodiments, R₂ is C₃ alkyl, such as n-propyl or isopropyl. In some embodiments, R₂ is C₄ alkyl, such as n-butyl or tert-butyl. In some embodiments, R₂ is C₅ alkyl, such as n-pentyl, sec-pentyl, or iso-pentyl. In some embodiments, R₂ is C₆ alkyl, such as n-hexyl.

In some embodiments, R₃ is halo. In some embodiments, R₃ is F, Cl, Br, or I. In some embodiments, R₃ is F. In some embodiments, R₃ is Br. In some embodiments, R₃ is Cl. In some embodiments, R₃ is I. In some embodiments, R₃ is C₁-C₆ alkyl. In some embodiments, R₃ is C₁-C₃ alkyl. In some embodiments, R₃ is C₁ alkyl (i.e., methyl). In some embodiments, R₃ is C₂ alkyl (i.e., ethyl). In some embodiments, R₃ is C₃ alkyl, such as n-propyl or isopropyl. In some embodiments, R₃ is C₄ alkyl, such as n-butyl, sec-butyl, or tert-butyl. In some embodiments, R₃ is C₅ alkyl, such as n-pentyl, sec-pentyl, or iso-pentyl. In some embodiments, R₃ is C₆ alkyl, such as n-hexyl.

In some embodiments, R₄ is H. In some embodiments, R₄ is halo such as F, Cl, Br, or I. In some embodiments, R₄ is F. In some embodiments, R₄ is Cl. In some embodiments, R₄ is Br. In some embodiments, R₄ is I.

In some embodiments, Q is C₁-C₆ alkyl optionally substituted by 1-5 substituents independently selected from hydroxy, —O(C₁-C₆ alkyl), and halo. In some embodiments, Q is C₁-C₆ alkyl optionally substituted by 1-3 substituents independently selected from hydroxy, —O(C₁-C₆ alkyl), and halo. In some embodiments, Q is C₁-C₃ alkyl optionally substituted by 1-5 substituents independently selected from hydroxy, —O(C₁-C₆ alkyl), and halo. In some embodiments, Q is C₁ alkyl (i.e., methyl) optionally substituted by 1-5 substituents independently selected from hydroxy,
—O(C₁-C₆ alkyl), and halo. In some embodiments, Q is C₂ alkyl (i.e., ethyl) optionally substituted by 1-5 substituents independently selected from hydroxy, —O(C₁-C₆ alkyl), and halo. In some embodiments, Q is C₃ alkyl, such as n-propyl or isopropyl, optionally substituted by 1-5 substituents independently selected from hydroxy, —O(C₁-C₆ alkyl), and halo. In some embodiments, Q is C₄ alkyl, such as n-butyl, sec-butyl, or tert-butyl, optionally substituted by 1-5 substituents independently selected from hydroxy, —O(C₁-C₆ alkyl), and halo. In some embodiments, Q is C₅ alkyl, such as n-pentyl, sec-pentyl, or iso-pentyl, optionally substituted by 1-5 substituents independently selected from hydroxy, —O(C₁-C₆ alkyl), and halo. In some embodiments, Q is C₆ alkyl, such as n-hexyl, optionally substituted by 1-5 substituents independently selected from hydroxy, —O(C₁-C₆ alkyl), and halo. In some embodiments, Q is C₁-C₆ alkyl optionally substituted by 1-5 substituents independently selected from hydroxy, —O(C₁-C₃ alkyl), and halo (such as F, Cl, or Br).

In some embodiments, Q is C₃-C₆ cycloalkyl optionally substituted by 1-5 substituents independently selected from hydroxy, —O(C₁-C₆ alkyl), and halo. In some embodiments, Q is C₃-C₆ cycloalkyl optionally substituted by 1-3 substituents independently selected from hydroxy, —O(C₁-C₆ alkyl), and halo. In some embodiments, Q is C₃ cycloalkyl (i.e., cyclopropyl) optionally substituted by 1-5 substituents independently selected from hydroxy, —O(C₁-C₆ alkyl), and halo. In some embodiments, Q is C₄ cycloalkyl (i.e., cyclobutyl) optionally substituted by 1-5 substituents independently selected from hydroxy, —O(C₁-C₆ alkyl), and halo. In some embodiments, Q is C₅ cycloalkyl (i.e., cyclopentyl) optionally substituted by 1-5 substituents independently selected from hydroxy, —O(C₁-C₆ alkyl), and halo. In some embodiments, Q is C₆ cycloalkyl (i.e., cyclohexyl) optionally substituted by 1-5 substituents independently selected from hydroxy, —O(C₁-C₆ alkyl), and halo. In some embodiments, Q is C₃-C₆ cycloalkyl optionally substituted by 1-5 substituents independently selected from hydroxy, —O(C₃-C₆ alkyl) such as methoxy or ethoxy, and halo (such as F, Cl, or Br).

In some embodiments, X₁ is CH; R₁ is C₃-C₆ alkyl optionally substituted by 1-3 substituents independently selected from halo, hydroxy, and —O(C₁-C₆ alkyl); R₂ is H; R₃ is halo;
R₄ is H; and Q is C₁-C₃ alkyl or C₃-C₆ cycloalkyl, each of which is optionally substituted by 1-3 substituents independently selected from hydroxy and —O(C₁-C₃ alkyl).

In some embodiments, R₁ is isopropyl or tert-butyl; R₃ is Cl; and Q is —CH₃, —CH₂CH₂OH, —(CH₂)₃O CH(CH₃)₂, or cyclopropyl.

In some embodiments, the compound of Formula (I), or a pharmaceutically acceptable salt thereof, is any one of the compounds, or a pharmaceutically acceptable salt thereof, provided in Table 1.

TABLE 1
Compounds of Formula (I).

| Nomenclature | Structure |
|---|---|
| 2-(4-Amino-1-tert-butyl-pyrazolo[3,4- d]pyrimidin-3-yl)-3-chloro-N-methyl-1H- indole-6-carboxamide (“Compound A”) | 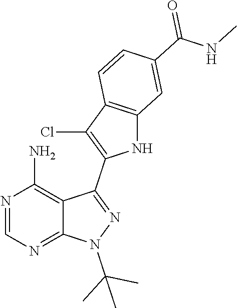 |
| 2-(4-Amino-1-isopropyl-pyrazolo[3,4- d]pyrimidin-3-yl)-3-chloro-N-methyl-1H- indole-6-carboxamide | 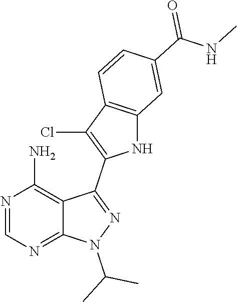 |
| 2-(4-Amino-1-tert-butyl-pyrazolo[3,4- d]pyrimidin-3-yl)-3-chloro-N-(2- hydroxyethyl)-1H-indole-6-carboxamide | 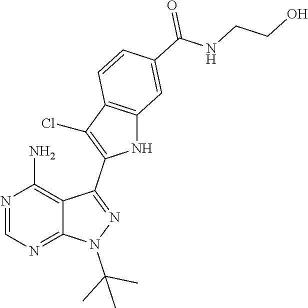 |
| 2-{4-Amino-1-tert-butyl-1H-pyrazolo[3,4- d]pyrimidin-3-yl}-3-chloro-N-cyclopropyl- 1H-indole-6-carboxamide | 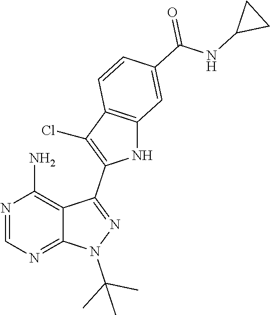 |
| 2-(4-Amino-1-tert-butyl-pyrazolo[3,4- d]pyrimidin-3-yl)-3-chloro-N-(3- isopropoxypropyl)-1H-indole-6- carboxamide | 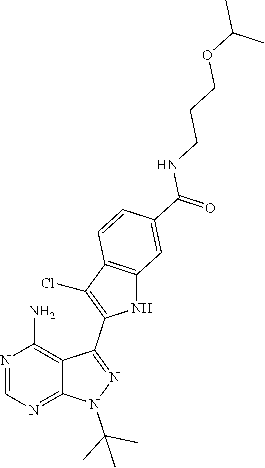 |

In other embodiments, the compound of Formula (I), or a pharmaceutically acceptable salt thereof, is any one of the compounds, or a pharmaceutically acceptable salts thereof, provided in Table 2.

TABLE 2
Additional Compounds of Formula (I).

| Nomenclature | Structure |
|---|---|
| 2-(4-Amino-1-(tert-butyl)-1H-pyrazolo[3,4- d]pyrimidin-3-yl)-3-bromo-N-methyl-1H- indole-6-carboxamide | 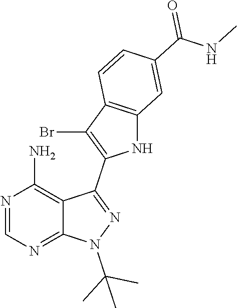 |
| 2-(4-Amino-1-tert-butyl-pyrazolo[3,4- d]pyrimidin-3-yl)-3-chloro-N-(2- methoxyethyl)-1H-indole-6-carboxamide | 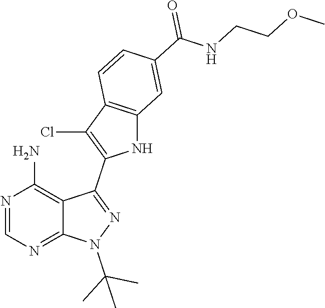 |
| 2-(4-Amino-1-tert-butyl-pyrazolo[3,4- d]pyrimidin-3-yl)-3-chloro-N-(3- methoxypropyl)-1H-indole-6-carboxamide | 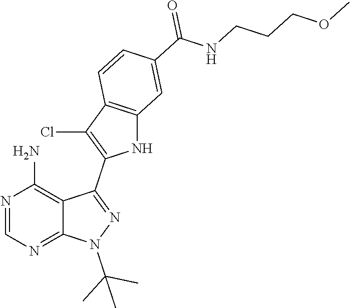 |
| 2-(4-Amino-1-tert-butyl-pyrazolo[3,4- d]pyrimidin-3-yl)-3-fluoro-N-methyl-1H- indole-6-carboxamide | 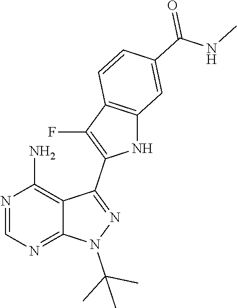 |
| 2-[4-Amino-1-(2- hydroxyethyl)pyrazolo[3,4-d]pyrimidin-3- yl]-3-chloro-N-methyl-1H-indole-6- carboxamide | 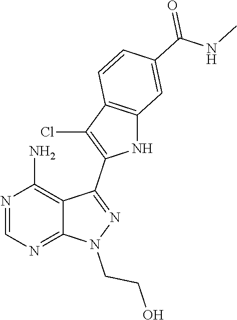 |
| 2-[4-Amino-1-(3- methoxypropyl)pyrazolo[3,4-d]pyrimidin-3- yl]-3-chloro-N-methyl-1H-indole-6- carboxamide | 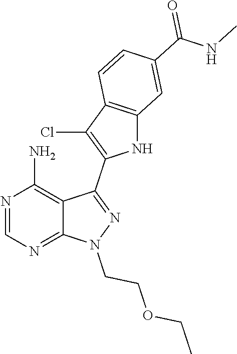 |
| 2-(4-Amino-1-methyl-pyrazolo[3,4- d]pyrimidin-3-yl)-3-chloro-N-methyl-1H- indole-6-carboxamide | 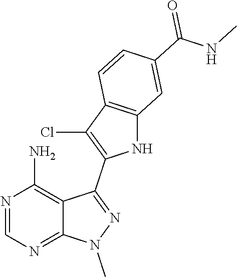 |
| 2-{4-Amino-1-tert-butyl-1H-pyrazolo[3,4- d]pyrimidin-3-yl}-3-chloro-N-methyl-1H- pyrrolo[2,3-b]pyridine-6-carboxamide | 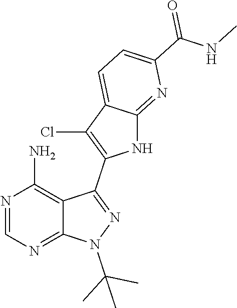 |
| 2-{4-Amino-1-tert-butyl-1H-pyrazolo[3,4- d]pyrimidin-3-yl}-3-chloro-N-(propan-2-yl)- 1H-indole-6-carboxamide | 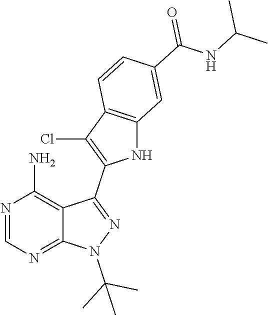 |
| 2-{4-Amino-1-tert-butyl-1/-pyrazolo[3,4- d]pyrimidin-3-yl}-3-chloro-N-ethyl-1H- indole-6-carboxamide | 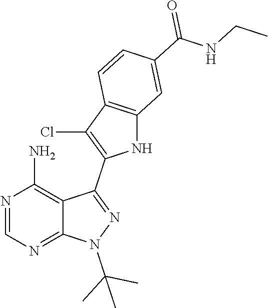 |
| 2-{4-Amino-1-cyclobutyl-1H-pyrazolo[3,4- d]pyrimidin-3-yl}-3-chloro-N-methyl-1H- indole-6-carboxamide | 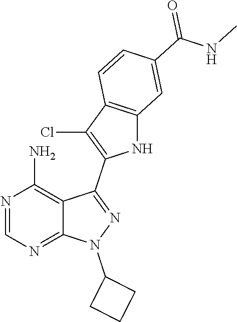 |
| 2-(4-Amino-1-cyclohexyl-1H-pyrazolo[3,4- d]pyrimidin-3-yl)-3-chloro-N-methyl-1H- indole-6-carboxamide | 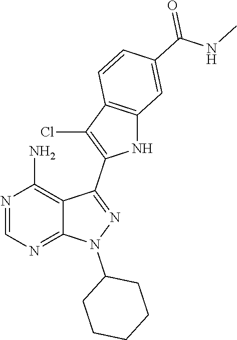 |
| 2-{4-Amino-1-tert-butyl-1H-pyrazolo[3,4- d]pyrimidin-3-yl}-3,5-dichloro-N-methyl- 1H-indole-6-carboxamide | 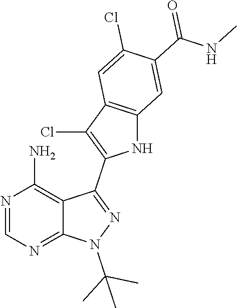 |
| 2-{4-Amino-1-tert-butyl-1H-pyrazolo[3,4- d]pyrimidin-3-yl}-3-chloro-N-(3,3,3- trifluoropropyl)-1H-indole-6-carboxamide | 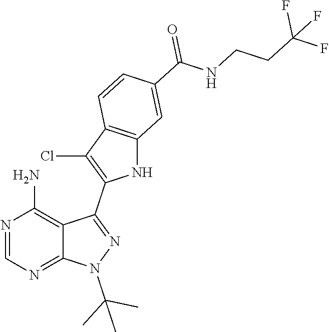 |
| 2-(4-Amino-1-(tert-butyl)-1H-pyrazolo[3,4- d]pyrimidin-3-yl)-N,3-dimethyl-1H-indole- 6-carboxamide | 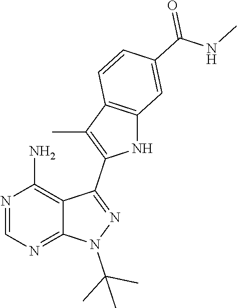 |
| 2-(4-Amino-1-(tert-butyl)-1H-pyrazolo[3,4- d]pyrimidin-3-yl)-3-chloro-N,1-dimethyl- 1H-indole-6-carboxamide | 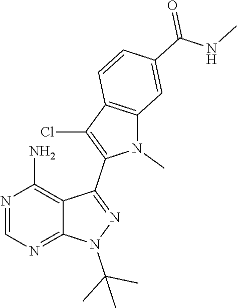 |
| 2-{4-Amino-1-ethyl-1H-pyrazolo[3,4- d]pyrimidin-3-yl}-3-chloro-N-methyl-1H- indole-6-carboxamide | 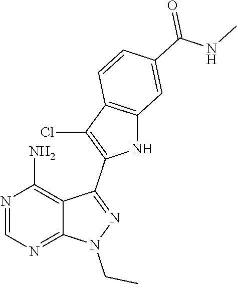 |
| 2-{4-Amino-1-cyclopropyl-1H- pyrazolo[3,4-d]pyrimidin-3-yl}-3-chloro-N- methyl-1H-indole-6-carboxamide | 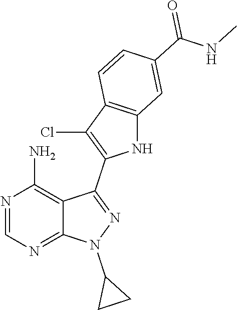 |
| 2-{4-Amino-1-cyclopentyl-1H-pyrazolo[3,4- d]pyrimidin-3-yl}-3-chloro-N-methyl-1H- indole-6-carboxamide | 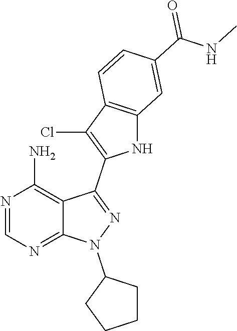 |
| 2-{4-Amino-1-tert-butyl-1H-pyrazolo[3,4- d]pyrimidin-3-yl}-3-chloro-N-propyl-1H- indole-6-carboxamide | 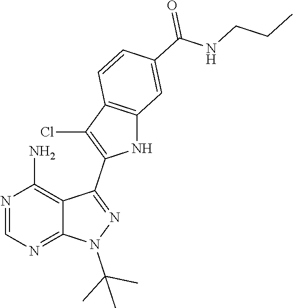 |
| 2-[4-Amino-1-(2,2,2-trifluoroethyl)-1H- pyrazolo[3,4-d]pyrimidin-3-yl]-3-chloro-N- ethyl-1H-indole-6-carboxamide | 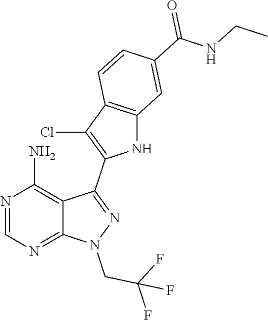 |
| 2-[4-Amino-1-(2,2,2-trifluoroethyl)-1H- pyrazolo[3,4-d]pyrimidin-3-yl]-3-chloro-N- methyl-1H-indole-6-carboxamide | 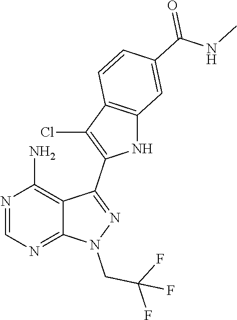 |
| 2-[4-Amino-1-(2,2,2-trifluoroethyl)-1H- pyrazolo[3,4-d]pyrimidin-3-yl]-3-chloro-N- cyclopropyl-1H-indole-6-carboxamide | 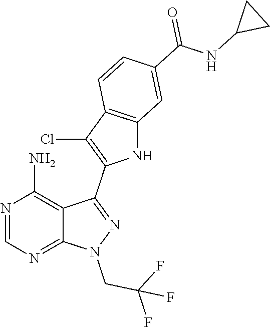 |
| 2-[4-Amino-1-(3-hydroxycyclopentyl)-1H- pyrazolo[3,4-d]pyrimidin-3-yl]-3-chloro-N- cyclopropyl-1H-indole-6-carboxamide | 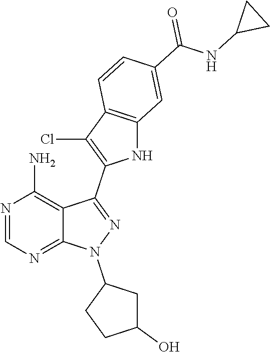 |

All compounds of Formula (I) or any variation thereof as described herein which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

The compounds of Formula (I), or a pharmaceutically acceptable salt thereof, can be prepared according to the procedures described in PCT Publication No. WO 2017/178844, the disclosure of which is incorporated herein by reference.

In some embodiments, the compounds of Formula (I), or a pharmaceutically acceptable salt thereof, show similar potency against both wild type RET kinase and RET kinase mutants, for example solvent front RET kinase mutants (such as G810 mutant RET kinases). In some embodiments, the potency is determined by IC₅₀ values, for example by the methods described in Examples 1 and 2. In some embodiments, the compounds of Formula (I), or a pharmaceutically acceptable salt thereof, promote reduced cell survival against cell lines expressing both wild type RET kinase and RET kinase mutants (such as cell lines harboring a G810 mutant RET kinase). In some embodiments, cell survival or cell viability is determined using a luminescent assay, such as described in Example 2.

### Pharmaceutical Compositions

In another aspect, provided herein are pharmaceutical compositions of the compounds of Formula (I) or a pharmaceutically acceptable salt thereof. Thus, the present disclosure includes pharmaceutical compositions comprising a compound of Formula (I), or a pharmaceutically acceptable salt thereof, and a pharmaceutically acceptable carrier or excipient. Pharmaceutical compositions according to the disclosure may take a form suitable for oral, buccal, sublingual, parenteral (subcutaneous, intramuscular, intravenous, or intrathecal), nasal, topical, vaginal, rectal, intracerebral, intradermal, intravitreal, intraosseous infusion, intraperitoneal, or inhalation administration. Pharmaceutical compositions of the present disclosure comprise a compound of Formula (I), or a pharmaceutically acceptable salt thereof, and a pharmaceutically acceptable carrier, diluent, or excipient.

A compound described herein can be used in the preparation of a pharmaceutical composition by combining the compound as an active ingredient with a pharmaceutically acceptable excipient. Some examples of materials which can serve as pharmaceutically acceptable excipients include: sugars, such as lactose, glucose and sucrose; starches, such as corn starch and potato starch; cellulose and its derivatives, such as sodium carboxymethyl cellulose, ethyl cellulose and cellulose acetate; surfactants, such as polysorbate 80 (i.e., Tween 80); powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Pharmaceutical formulations may be prepared by known pharmaceutical methods. Suitable formulations can be found in, for example, Remington: The Science and Practice of Pharmacy, Lippincott Williams & Wilkins, 21^{st} ed. (2005), which is incorporated herein by reference.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol and the like; and metal chelating agents, such as citric acid, ethylenediamine tetraacetic acid (EDTA), sorbitol, tartaric acid, phosphoric acid and the like.

The pharmaceutical compositions may conveniently be presented in unit dosage form and may be prepared by any methods well known in the art of pharmacy. The amount of active ingredient which can be combined with a carrier material to produce a single dosage form will vary depending upon the subject being treated and the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%.

In certain embodiments, a pharmaceutical composition of the present disclosure comprises an excipient selected from the group consisting of cyclodextrins, liposomes, micelle forming agents, e.g., bile acids and polymeric carriers, e.g., polyesters and polyanhydrides; and a compound of Formula (I) or a pharmaceutically acceptable salt thereof. In certain embodiments, the pharmaceutical composition renders orally bioavailable a compound of Formula (I) or a pharmaceutically acceptable salt thereof.

Pharmaceutical compositions of the disclosure suitable for oral administration may be in the form of capsules, cachets, pills, tablets, lozenges (using a flavored basis, usually sucrose and acacia or tragacanth), powders, granules or as a solution or a suspension in an aqueous or non-aqueous liquid or as an oil-in-water or water-in-oil liquid emulsion or as an elixir or syrup or as pastilles (using an inert base, such as gelatin and glycerin or sucrose and acacia) and/or as mouth washes and the like, each containing a predetermined amount of a compound of Formula (I), or a pharmaceutically acceptable salt thereof, as an active ingredient. A compound of Formula (I), or a pharmaceutically acceptable salt thereof, may also be administered as a bolus, electuary, or paste.

In solid dosage forms of the disclosure for oral administration (capsules, tablets, pills, dragees, powders, granules and the like), the active ingredient is mixed with one or more pharmaceutically-acceptable carriers, such as sodium citrate or dicalcium phosphate and/or any of the following: fillers or extenders, such as starches, lactose, sucrose, glucose, mannitol and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates and sodium carbonate; solution retarding agents, such as paraffin; absorption accelerators, such as quaternary ammonium compounds; wetting agents, such as, for example, cetyl alcohol, glycerol monostearate and non-ionic surfactants; absorbents, such as kaolin and bentonite clay; lubricants, such a talc, calcium stearate, magnesium stearate, solid polyethylene glycols, sodium lauryl sulfate and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter or by incorporating sterilizing agents in the form of sterile solid compositions that can be dissolved in sterile water or some other sterile injectable medium immediately before use. These compositions may also optionally contain opacifying agents and may be of a composition that they release the active ingredient(s) only or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of Formula (I), or a pharmaceutically acceptable salt thereof, include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth and mixtures thereof.

Pharmaceutical compositions of the disclosure for rectal or vaginal administration may be presented as a suppository, which may be prepared by mixing one or more compounds of the disclosure with one or more suitable nonirritating excipients or carriers comprising, for example, cocoa butter, polyethylene glycol, a suppository wax or a salicylate and which is solid at room temperature, but liquid at body temperature and, therefore, will melt in the rectum or vaginal cavity and release the active compound.

Dosage forms for the topical or transdermal administration of a compound of this disclosure include powders, sprays, ointments, pastes, creams, lotions, gels, solutions, patches and inhalants. The active compound (i.e., a compound of Formula (I) or a pharmaceutically acceptable salt thereof) may be mixed under sterile conditions with a pharmaceutically-acceptable carrier and with any preservatives, buffers or propellants which may be required.

The ointments, pastes, creams, and gels may contain, in addition to a compound of Formula (I), or a pharmaceutically acceptable salt thereof, excipients such as animal and vegetable fats, oils, waxes, paraffins, starch, tragacanth, cellulose derivatives, polyethylene glycols, silicones, bentonites, silicic acid, talc and zinc oxide or mixtures thereof.

Powders and sprays can contain, in addition to a compound of Formula (I), or a pharmaceutically acceptable salt thereof, excipients such as lactose, talc, silicic acid, aluminum hydroxide, calcium silicates and polyamide powder or mixtures of these substances. Sprays can additionally contain customary propellants, such as chlorofluorohydrocarbons and volatile unsubstituted hydrocarbons, such as butane and propane.

Pharmaceutical compositions of this disclosure suitable for parenteral administration comprise one or more compounds of Formula (I), or a pharmaceutically acceptable salt thereof, in combination with one or more pharmaceutically-acceptable sterile isotonic aqueous or nonaqueous solutions, dispersions, suspensions or emulsions or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the disclosure include water, ethanol, polyols (such as glycerol, propylene glycol, polyethylene glycol and the like) and suitable mixtures thereof, vegetable oils, such as olive oil and injectable organic esters, such as ethyl oleate. Proper fluidity can be maintained, for example, by the use of coating materials, such as lecithin, by the maintenance of the required particle size in the case of dispersions and by the use of surfactants.

The pharmaceutical compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenyl sorbic acid and the like. It may also be desirable to include isotonic agents, such as sugars, sodium chloride and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

### Methods of Treatment

Compounds of Formula (I), or a pharmaceutically acceptable salt thereof, and pharmaceutical compositions comprising compounds of Formula (I), or a pharmaceutically acceptable salt thereof, may be used in methods of administration and treatment as provided herein. The compounds and pharmaceutical compositions may also be used in in vitro methods, such as in vitro methods of administering a compound or pharmaceutical composition to cells for screening purposes and/or for conducting quality control assays.

In one aspect, provided herein is a method for treating a cancer associated with RET kinase activity in a subject in need thereof, comprising administering to the subject an effective amount of a compound of Formula (I), or a pharmaceutically acceptable salt thereof, wherein the cancer has developed resistance following prior therapy and/or wherein the subject has a solvent front mutation in the RET kinase.

In another aspect, provided herein is a pharmaceutical composition for treating a subject with a cancer associated with RET kinase activity and wherein the cancer has developed resistance following prior therapy and/or wherein the subject has a solvent front mutation in the RET kinase, comprising a therapeutically effective amount of a compound of Formula (I) or a pharmaceutically acceptable salt thereof.

In a further aspect, provided herein is a method of inhibiting a mutant RET kinase, comprising contacting the mutant RET kinase with an effective amount of a compound of Formula (I) or a pharmaceutically acceptable salt thereof. In some embodiments, the method is an in vitro method. In other embodiments, the method is an in vivo method.

Also provided herein is a compound of Formula (I), or a pharmaceutically acceptable salt thereof, for use in a method disclosed herein for treating a cancer associated with RET kinase activity in a subject in need thereof.

In a further aspect, provided herein is use of a compound of Formula (I), or a pharmaceutically acceptable salt thereof, for the manufacture of a medicament for a method disclosed herein of treating a cancer associated with RET kinase activity in a subject in need thereof.

### Prior Therapy

In some embodiments, the cancer has developed resistance following prior therapy. In some embodiments, the prior therapy is a kinase inhibitor, immunotherapy, chemotherapy, surgery, or radiation. In some embodiments, the prior therapy is a combination of two or more of a kinase inhibitor, immunotherapy, chemotherapy, surgery, and radiation.

In some embodiments, the prior therapy is a kinase inhibitor. In some embodiments, the kinase inhibitor is a selective RET kinase inhibitor or a multikinase inhibitor.

In some embodiments, the kinase inhibitor is a selective RET kinase inhibitor. In some embodiments, the selective RET kinase inhibitor is selpercatinib or pralsetinib. In some embodiments, the selective RET kinase inhibitor is selpercatinib. In some embodiments, the selective RET kinase inhibitor is pralsetinib.

In some embodiments, the kinase inhibitor is a multikinase inhibitor. In some embodiments, the multikinase inhibitor is nintedanib, vandetanib, cabozantinib, lenvatinib, RXDX-105, sunitinib, sorafenib, alectinib, ponatinib, or regorafenib. In some embodiments, the multikinase inhibitor is nintedanib. In some embodiments, the multikinase inhibitor is vandetanib. In some embodiments, the multikinase inhibitor cabozantinib. In some embodiments, the multikinase inhibitor is lenvatinib. In some embodiments, the multikinase inhibitor is RXDX-105. In some embodiments, the multikinase inhibitor is sunitinib. In some embodiments, the multikinase inhibitor is sorafenib. In some embodiments, the multikinase inhibitor is alectinib. In some embodiments, the multikinase inhibitor is ponatinib. In some embodiments, the multikinase inhibitor is regorafenib.

In some embodiments, the prior therapy is immunotherapy. As used herein, immunotherapy refers to a type of treatment that utilizes the immune system of the subject to fight a disease such as cancer. Immunotherapy often involves stimulation of the immune system of the subject or supplementation of lab-derived immune system mimicking components to find and attack cancer cells. In some variations, immunotherapy includes anti-PD-1/PD-L1 and anti-CTLA-4 antibodies. In some embodiments, the prior therapy is chemotherapy. As used herein, chemotherapy refers to treatment of cancer that involves medicines or drugs to kill cancer cells. In some variations, chemotherapy includes doxorubicin, cisplatin, and 5-fluorouracil. In some embodiments, the prior therapy is surgery. As used herein, surgery refers to removal of the tumor and nearby tissue during an operation. Surgery is a local treatment that affects only one part of the body. In some variations, surgery includes open surgery and minimally invasive surgery. In some embodiments, the prior therapy is radiation. As used herein, radiation refers to treatment of cancer with high doses of radiation to kill cancer cells and shrink tumors. In some variations, radiation includes X-rays (i.e., from an external beam source) as well as brachytherapy, wherein radiation is placed inside the body of the subject.

In some embodiments, the prior therapy is a first-line therapy. As used herein, first-line therapy refers to the first treatment given for a disease. First-line therapy is often part of a standard set of treatments, such as surgery followed by chemotherapy and radiation. When used by itself, first-line therapy is the one accepted by the treating physician as the best treatment. In some embodiments, the prior therapy is a second-line therapy. As used herein, second-line therapy refers to treatment that is given when initial treatment (first-line therapy) does not work or has stopped working. In some embodiments, the prior therapy is a third-line therapy. As used herein, third-line therapy refers to treatment that is given when both initial treatment (first-line therapy) and subsequent treatment (second-line therapy) do not work or have stopped working. In some embodiments, the first-line, second-line, or third-line therapy is a kinase inhibitor, immunotherapy, chemotherapy, surgery, or radiation as described herein. In some embodiments, the prior therapy includes more than one of the prior therapies listed herein. In other embodiments, the prior therapy is limited to a single prior therapy as described herein.

### Cancer Type

In some embodiments, the cancer has developed resistance following prior therapy. In some embodiments, the cancer is a malignant neoplasm, a malignant tumor, or a solid tumor. In some embodiments, the cancer is a malignant neoplasm. In some embodiments, the cancer is a malignant tumor. In some embodiments, the cancer is a solid tumor. In some embodiments, the original cancer (e.g., a tumor) is not resistant to the prior therapy but has metastasized to a cancer this is resistant to the prior therapy.

In some embodiments, the cancer is leukemia, lung cancer, colon cancer, breast cancer, ovarian cancer, prostate cancer, liver cancer, pancreatic cancer, brain cancer, skin cancer, thyroid cancer, salivary gland cancer, endocrine cancer, urothelial cancer, uterine cancer, fallopian tube cancer, gastrointestinal cancer, or esophageal cancer. In some embodiments, the cancer is leukemia. In some embodiments, the cancer is lung cancer. In some embodiments, the cancer is colon cancer. In some embodiments, the cancer is breast cancer. In some embodiments, the cancer is ovarian cancer. In some embodiments, the cancer is prostate cancer. In some embodiments, the cancer is liver cancer. In some embodiments, the cancer is pancreatic cancer. In some embodiments, the cancer is brain cancer. In some embodiments, the cancer is skin cancer. In some embodiments, the cancer is thyroid cancer. In some embodiments, the cancer is salivary gland cancer. In some embodiments, the cancer is endocrine cancer. In some embodiments, the cancer is urothelial cancer. In some embodiments, the cancer is uterine cancer. In some embodiments, the cancer is fallopian tube cancer. In some embodiments, the cancer is gastrointestinal cancer. In some embodiments, the cancer is esophageal cancer.

In some embodiments, the cancer is medullary thyroid cancer, non-small cell lung cancer, lung carcinosarcoma, lung adenocarcinoma, atypical lung carcinoid, multiple endocrine neoplasia type 2, ovarian epithelial carcinoma, uterine carcinosarcoma, fallopian tube adenocarcinoma, ovarian epithelial carcinoma, chronic myelomonocytic leukemia (CMML), melanoma, basal cell carcinoma, Merkel cell carcinoma, salivary gland adenocarcinoma, papillary thyroid carcinoma (PTC), anaplastic thyroid carcinoma, meningioma, esophageal adenocarcinoma, gastric adenocarcinoma, ureter urothelial carcinoma, duodenal adenocarcinoma, or colorectal adenocarcinoma. In some embodiments, the cancer is medullary thyroid cancer. In some embodiments, the cancer is non-small cell lung cancer. In some embodiments, the cancer is lung carcinosarcoma. In some embodiments, the cancer is lung adenocarcinoma. In some embodiments, the cancer is atypical lung carcinoid. In some embodiments, the cancer is multiple endocrine neoplasia type 2. In some embodiments, the cancer is uterine carcinosarcoma. In some embodiments, the cancer is fallopian tube adenocarcinoma. In some embodiments, the cancer is ovarian epithelial carcinoma. In some embodiments, the cancer is chronic myelomonocytic leukemia (CMML). In some embodiments, the cancer is melanoma. In some embodiments, the cancer is basal cell carcinoma. In some embodiments, the cancer is Merkel cell carcinoma. In some embodiments, the cancer is salivary gland adenocarcinoma. In some embodiments, the cancer is papillary thyroid carcinoma (PTC). In some embodiments, the cancer is anaplastic thyroid carcinoma. In some embodiments, the cancer is meningioma. In some embodiments, the cancer is esophageal adenocarcinoma. In some embodiments, the cancer is gastric adenocarcinoma. In some embodiments, the cancer is ureter urothelial carcinoma. In some embodiments, the cancer is duodenal adenocarcinoma. In some embodiments, the cancer is colorectal adenocarcinoma.

In some embodiments, the cancer shows evidence of activating and/or oncogenic RET alterations. In some embodiments, the cancer is associated with aberrant RET kinase activity. Aberrant RET kinase activity refers to a level of RET kinase activity that is significantly different (i.e., higher or lower) than the level of RET kinase activity in a subject that does not have a cancer associated with RET kinase activity or in a healthy subject (for example, a subject that does not have cancer).

### Mutations of RET Kinase

In some embodiments, the resistance developed by the cancer is due to a solvent front mutation in the RET kinase. In some embodiments, the solvent front mutation is at G810 in the amino acid sequence of the RET kinase. In some embodiments, the solvent front mutation is G810A, G810C, G810R, G810V, or G810S. In some embodiments, the solvent front mutation is G810A. In some embodiments, the solvent front mutation is G810C. In some embodiments, the solvent front mutation is G810R. In some embodiments, the solvent front mutation is G810V. In some embodiments, the solvent front mutation is G810S.

In some embodiments, the amino acid sequence of RET kinase comprises the amino acid sequence of wild type RET kinase (RET WT). In some embodiments, the amino acid sequence of RET kinase comprises the amino acid sequence of the cytoplasmic domain of RET WT. In some embodiments, the cytoplasmic domain of RET WT comprises amino acids 658-1114 of accession number NP_066124.1. In some embodiments, the amino acid sequence of the cytoplasmic domain of RET kinase comprises the sequence of SEQ ID NO: 1:

(SEQ ID NO: 1)
HCYHKFAHKPPISSAEMTFRRPAQAFPVSYSSSGARRPSLDSMENQVSV
DAFKILEDPKWEFPRKNLVLGKTLGEGEFGKVVKATAFHLKGRAGYTTV
AVKMLKENASPSELRDLLSEFNVLKQVNHPHVIKLYGACSQDGPLLLIV
EYAKYGSLRGFLRESRKVGPGYLGSGGSRNSSSLDHPDERALTMGDLIS
FAWQISQGMQYLAEMKLVHRDLAARNILVAEGRKMKISDFGLSRDVYEE
DSYVKRSQGRIPVKWMAIESLFDHIYTTQSDVWSFGVLLWEIVTLGGNP
YPGIPPERLFNLLKTGHRMERPDNCSEEMYRLMLQCWKQEPDKRPVFAD
ISKDLEKMMVKRRDYLDLAASTPSDSLIYDDGLSEEETPLVDCNNAPLP
RALPSTWIENKLYGMSDPNWPGESPVPLTRADGTNTGFPRYPNDSVYAN
WMLSPSAAKLMDTFDS.

In some embodiments, the amino acid sequence of RET kinase comprises at least about 80% sequence identity, such as at least about 80%, 85%, 90%, 95%, 97%, 98%, or 99% sequence identity, to SEQ ID NO: 1. In some embodiments, the amino acid sequence of RET kinase comprises about 80%, about 85%, about 90%, about 95%, about 97%, about 98%, or about 99% sequence identity to SEQ ID NO: 1. In some embodiments, the amino acid sequence of RET kinase comprises the sequence of SEQ ID NO: 1.

In some embodiments, the amino acid sequence of RET kinase comprises the amino acid sequence of the full-length RET WT. In some embodiments, the full-length RET WT comprises amino acids 1-1114 of accession number NP_066124.1. In some embodiments, the amino acid sequence of the full-length RET kinase comprises the sequence of SEQ ID NO: 2:

(SEQ ID NO: 2)
MAKATSGAAGLRLLLLLLLPLLGKVALGLYFSRDAYWEKLYVDQAAGTP
LLYVHALRDAPEEVPSFRLGQHLYGTYRTRLHENNWICIQEDTGLLYLN
RSLDHSSWEKLSVRNRGFPLLTVYLKVFLSPTSLREGECQWPGCARVYF
SFFNTSFPACSSLKPRELCFPETRPSFRIRENRPPGTFHQFRLLPVQFL
CPNISVAYRLLEGEGLPFRCAPDSLEVSTRWALDREQREKYELVAVCTV
HAGAREEVVMVPFPVTVYDEDDSAPTFPAGVDTASAVVEFKRKEDTVVA
TLRVFDADVVPASGELVRRYTSTLLPGDTWAQQTFRVEHWPNETSVQAN
GSFVRATVHDYRLVLNRNLSISENRTMQLAVLVNDSDFQGPGAGVLLLH
FNVSVLPVSLHLPSTYSLSVSRRARRFAQIGKVCVENCQAFSGINVQYK
LHSSGANCSTLGVVTSAEDTSGILFVNDTKALRRPKCAELHYMVVATDQ
QTSRQAQAQLLVTVEGSYVAEEAGCPLSCAVSKRRLECEECGGLGSPTG
RCEWRQGDGKGITRNFSTCSPSTKTCPDGHCDVVETQDINICPQDCLRG
SIVGGHEPGEPRGIKAGYGTCNCFPEEEKCFCEPEDIQDPLCDELCRTV
IAAAVLFSFIVSVLLSAFCIHCYHKFAHKPPISSAEMTFRRPAQAFPVS
YSSSGARRPSLDSMENQVSVDAFKILEDPKWEFPRKNLVLGKTLGEGEF
GKVVKATAFHLKGRAGYTTVAVKMLKENASPSELRDLLSEFNVLKQVNH
PHVIKLYGACSQDGPLLLIVEYAKYGSLRGFLRESRKVGPGYLGSGGSR
NSSSLDHPDERALTMGDLISFAWQISQGMQYLAEMKLVHRDLAARNILV
AEGRKMKISDFGLSRDVYEEDSYVKRSQGRIPVKWMAIESLFDHIYTTQ
SDVWSFGVLLWEIVTLGGNPYPGIPPERLFNLLKTGHRMERPDNCSEEM
YRLMLQCWKQEPDKRPVFADISKDLEKMMVKRRDYLDLAASTPSDSLIY
DDGLSEEETPLVDCNNAPLPRALPSTWIENKLYGMSDPNWPGESPVPLT
RADGTNTGFPRYPNDSVYANWMLSPSAAKLMDTFDS

In some embodiments, the amino acid sequence of RET kinase comprises at least about 80% sequence identity, such as at least about 80%, 85%, 90%, 95%, 97%, 98%, or 99% sequence identity, to SEQ ID NO: 2. In some embodiments, the amino acid sequence of RET kinase comprises about 80%, about 85%, about 90%, about 95%, about 97%, about 98%, or about 99% sequence identity to SEQ ID NO: 2. In some embodiments, the amino acid sequence of RET kinase comprises the sequence of SEQ ID NO: 2.

In some embodiments, the solvent front mutation is identified by a detection method comprising evaluating circulating tumor DNA and/or evaluating a tissue biopsy. Circulating tumor DNA is cell free DNA that is released into the bloodstream as dead cancer cells are broken down. In some embodiments, circulating tumor DNA comprises no more than 200 nucleotides. In some embodiments, the solvent front mutation is identified by a detection method comprising evaluating circulating tumor DNA and evaluating a tissue biopsy. In some embodiments, the solvent front mutation is identified by a detection method comprising evaluating circulating tumor DNA or evaluating a tissue biopsy. In some embodiments, the solvent front mutation is identified by a detection method comprising evaluating circulating tumor DNA. In some embodiments, the solvent front mutation is identified by a detection method comprising evaluating a tissue biopsy.

In some embodiments, the solvent front mutation is identified by a detection method that comprises sequencing.

In some embodiments, the original cancer (e.g., a tumor) does not have a solvent front mutation but has metastasized to a cancer that has a solvent front mutation.

In some embodiments, the RET kinase further comprises a RET fusion translocation and/or a mutation at the RET gatekeeper residue V804. In some embodiments, the RET kinase further comprises a RET fusion translocation and a mutation at the RET gatekeeper residue V804. In some embodiments, the RET kinase further comprises a RET fusion translocation or a mutation at the RET gatekeeper residue V804. In some embodiments, the RET kinase further comprises a RET fusion translocation. In some embodiments, the RET fusion translocation is KIF5B-RET or CCDC6-RET. In some embodiments, the RET fusion translocation is KIF5B-RET. In some embodiments, the RET fusion translocation is CCDC6-RET. In some embodiments, the RET kinase further comprises a mutation at the RET gatekeeper residue V804. In some embodiments, the RET gatekeeper residue V804 is RET^{V804M}.

In some embodiments, the RET fusion translocation is identified by a detection method comprising evaluating circulating tumor DNA and/or evaluating a tissue biopsy. In some embodiments, the RET fusion translocation is identified by a detection method comprising evaluating circulating tumor DNA and evaluating a tissue biopsy. In some embodiments, the RET fusion translocation is identified by a detection method comprising evaluating circulating tumor (cell-free) DNA or evaluating a tissue biopsy. In some embodiments, the RET fusion translocation is identified by a detection method comprising evaluating circulating tumor DNA. In some embodiments, the RET fusion translocation is identified by a detection method comprising evaluating a tissue biopsy. In some embodiments, the RET fusion translocation is identified by a detection method that comprises sequencing.

In some embodiments, the mutation at the RET gatekeeper residue V804 in the RET kinase is identified by a detection method comprising evaluating circulating tumor DNA and/or evaluating a tissue biopsy. In some embodiments, the mutation at the RET gatekeeper residue V804 in the RET kinase is identified by a detection method comprising evaluating circulating tumor DNA and evaluating a tissue biopsy. In some embodiments, the mutation at the RET gatekeeper residue V804 in the RET kinase is identified by a detection method comprising evaluating circulating tumor DNA or evaluating a tissue biopsy. In some embodiments, the mutation at the RET gatekeeper residue V804 in the RET kinase is identified by a detection method comprising evaluating circulating tumor DNA. In some embodiments, the mutation at the RET gatekeeper residue V804 in the RET kinase is identified by a detection method comprising evaluating a tissue biopsy. In some embodiments, the RET gatekeeper residue V804 in the RET kinase is identified by a detection method that comprises sequencing.

### Dosing and Method of Administration

The compounds of Formula (I), or a pharmaceutically acceptable salt thereof, or the pharmaceutical composition of Formula (I), or a pharmaceutically acceptable salt thereof, described herein may be administered to a subject by any suitable route of administration.

In some embodiments, the compound of Formula (I), or a pharmaceutically acceptable salt thereof, or the pharmaceutical composition of Formula (I), or a pharmaceutically acceptable salt thereof, described herein is administered to the subject by oral, buccal, sublingual, parenteral (subcutaneous, intramuscular, intravenous, or intrathecal), nasal, topical, vaginal, rectal, intracerebral, intradermal, intravitreal, intraosseous infusion, intraperitoneal, or inhalation administration. In some embodiments, the compound of Formula (I), or a pharmaceutically acceptable salt thereof, or the pharmaceutical composition of Formula (I), or a pharmaceutically acceptable salt thereof, described herein is administered to the subject by oral administration. In some embodiments, the compound of Formula (I), or a pharmaceutically acceptable salt thereof, or the pharmaceutical composition of Formula (I), or a pharmaceutically acceptable salt thereof, described herein is administered to the subject by intravenous, subcutaneous, or intramuscular administration. In some embodiments, the compound of Formula (I), or a pharmaceutically acceptable salt thereof, or the pharmaceutical composition of Formula (I), or a pharmaceutically acceptable salt thereof, described herein is administered to the subject by intravenous administration. In some embodiments, the compound of Formula (I), or a pharmaceutically acceptable salt thereof, or the pharmaceutical composition of Formula (I), or a pharmaceutically acceptable salt thereof, described herein is administered to the subject by subcutaneous administration. In some embodiments, the compound of Formula (I) or composition described herein is administered to the subject by intramuscular administration.

Regardless of the route of administration selected, the compounds of Formula (I), or a pharmaceutically acceptable salt thereof, or the pharmaceutical composition of Formula (I), or a pharmaceutically acceptable salt thereof, are formulated into pharmaceutically acceptable dosage forms by conventional methods known to those of skill in the art.

Actual dosage levels of the active ingredients in the pharmaceutical compositions of this disclosure may be varied so as to obtain an amount of the active ingredient that is effective to achieve the desired therapeutic response for a particular patient, composition and mode of administration, without being toxic to the patient.

The selected dosage level will depend upon a variety of factors including the activity of the particular compound of the present disclosure employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the subject being treated, and like factors well known in the medical arts. A daily, weekly or monthly dosage (or other time interval) can be used.

A physician or veterinarian having ordinary skill in the art can determine and prescribe the effective amount of the pharmaceutical composition required. For example, the physician or veterinarian could start doses of the compounds of Formula (I), or a pharmaceutically acceptable salt thereof, employed in the pharmaceutical composition at levels lower than that required to achieve the desired therapeutic effect and then gradually increasing the dosage until the desired effect is achieved.

In general, a suitable daily dose of a compound of the disclosure will be that amount of the compound that is the lowest dose effective to produce a therapeutic effect (e.g., inhibit a mutant RET kinase). Such an effective dose will generally depend upon the factors described above. Generally, doses of the compounds of Formula (I), or a pharmaceutically acceptable salt thereof, for a subject, when used for the indicated effects, will range from about 0.0001 to about 100 mg per kg of body weight per day. Preferably, the daily dosage will range from about 0.001 to about 50 mg of compound per kg of body weight, and even more preferably from about 0.01 to about 10 mg of compound per kg of body weight.

If desired, the effective daily dose of the active compound may be administered as two, three, four, five, six or more sub-doses administered separately at appropriate intervals throughout the day, optionally, in unit dosage forms.

When the compounds of Formula (I), or a pharmaceutically acceptable salt thereof, are administered as pharmaceuticals, to humans and animals, they can be given per se or as a pharmaceutical composition containing, for example, about 0.1% to 99.5% (more preferably, about 0.5% to 90%) of active ingredient in combination with a pharmaceutically acceptable carrier.

The compounds of Formula (I), or a pharmaceutically acceptable salt thereof, or the pharmaceutical compositions of Formula (I), or a pharmaceutically acceptable salt thereof, may be administered once, twice, three or four times daily, using any suitable mode described above. Also, administration or treatment with the compounds may be continued for a number of days; for example, commonly treatment would continue for at least about 7 days, about 14 days, or about 28 days, for one cycle of treatment. Treatment cycles are well known and are frequently alternated with resting periods of about 1 to 28 days, commonly about 7 days or about 14 days, between cycles. The treatment cycles, in certain embodiments, may also be continuous.

When administered orally, the total daily dosage for a human subject may be from about 1 to about 1,000 mg/day, from about 1,000 to about 2,000 mg/day, from about 10 to about 500 mg/day, from about 50 to about 300 mg/day, from about 75 to about 200 mg/day, or from about 100 to about 150 mg/day.

The daily dosage may also be described as a total amount of a compound described herein administered per dose or per day. Daily dosage of a compound may be from about 1 mg to about 4,000 mg, from about 2,000 to about 4,000 mg/day, from about 1 to about 2,000 mg/day, from about 1 to about 1,000 mg/day, from about 10 to about 500 mg/day, from about 20 to about 500 mg/day, from about 50 to about 300 mg/day, from about 75 to about 200 mg/day, or from about 15 to about 150 mg/day.

In certain embodiments, the method comprises administering to the subject an initial daily dose of about 1 to about 800 mg of a compound of Formula (I), or a pharmaceutically acceptable salt thereof, and increasing the dose by increments until clinical efficacy is achieved. Increments of about 5, 10, 25, 50 or 100 mg can be used to increase the dose. The dosage can be increased daily, every other day, twice per week or once per week.

The compound of Formula (I), or a pharmaceutically acceptable salt thereof, or composition described herein may be administered more than once, e.g., twice, three times, four times, five times, or more. In some embodiments, the duration of the treatment is up to about 36 months, such as about 1 month, about 2 months, about 3 months, about 6 months, about 9 months, about 12 months, about 15 months, about 18 months, about 21 months, about 24 months, about 27 months, about 30 months, about 33 months, or about 36 months. In some embodiments, the duration of treatment is further extended. In some embodiments, the duration of treatment is for the entire lifetime of the subject.

In some embodiments, the compound of Formula (I), or a pharmaceutically acceptable salt thereof, or composition described herein is administered to a subject in need thereof about once every day, about once every 2 days, about once every 3 days, about once every 4 days, about once every 5 days, about once every 6 days, about once every week, about once every two weeks, about once every three weeks, about once every 4 weeks, about once every 2 months, about once every 3 months, about once every 6 months, about once every 9 months, or about once every year.

### Subject

In some embodiments, a compound of Formula (I), or a salt thereof, or a pharmaceutical composition comprising a compound of Formula (I), or a pharmaceutically acceptable salt thereof, is administered to a subject having a cancer associated with RET kinase activity, wherein the cancer has developed resistance following prior therapy and/or wherein the subject has a solvent front mutation in the RET kinase. In some embodiments, the subject has a cancer associated with RET kinase activity, wherein the cancer has developed resistance following prior therapy, and the subject has a solvent front mutation in the RET kinase. In some embodiments, the subject has a cancer associated with RET kinase activity, wherein the cancer has developed resistance following prior therapy, or the subject has a solvent front mutation in the RET kinase. In some embodiments, the subject has a cancer associated with RET kinase activity, wherein the cancer has developed resistance following prior therapy. In some embodiments, the subject has a cancer associated with RET kinase activity, wherein the RET kinase has a solvent front mutation.

In some embodiments, the subject previously responded to the prior therapy. In some embodiments, the subject no longer responds to the prior therapy.

In some embodiments, the prior therapy is a kinase inhibitor, immunotherapy, chemotherapy, surgery, or radiation. In some embodiments, the prior therapy is a kinase inhibitor. In some embodiments, the kinase inhibitor is a selective RET kinase inhibitor or a multikinase inhibitor. In some embodiments, the kinase inhibitor is a selective RET kinase inhibitor. In some embodiments, the selective RET kinase inhibitor is selpercatinib or pralsetinib. In some embodiments, the kinase inhibitor is a multikinase inhibitor. In some embodiments, the multikinase inhibitor is nintedanib, vandetanib, cabozantinib, lenvatinib, RXDX-105, sunitinib, sorafenib, alectinib, ponatinib, or regorafenib. In some embodiments, the multikinase inhibitor is nintedanib. In some embodiments, the prior therapy is immunotherapy. In some embodiments, the prior therapy is chemotherapy. In some embodiments, the prior therapy is surgery. In some embodiments, the prior therapy is radiation.

In some embodiments, a compound of Formula (I), or a salt thereof, or a pharmaceutical composition comprising a compound of Formula (I), or a pharmaceutically acceptable salt thereof, is administered to a subject having a solvent front mutation in the RET kinase. In some embodiments, the solvent front mutation is at G810 in the amino acid sequence of the RET kinase. In some embodiments, the solvent front mutation is G810A, G810C, G810R, G810V, or G810S. In some embodiments, the amino acid sequence of the RET kinase comprises the amino acid sequence of SEQ ID NO: 1. In some embodiments, the RET kinase further comprises a RET fusion translocation and/or a mutation at the RET gatekeeper residue V804. In some embodiments, the RET fusion translocation is KIF5B-RET or CCDC6-RET. In some embodiments, the RET gatekeeper residue V804 is RET^{V804M}.

In some embodiments, the subject is a mammal. In some embodiments, the subject is a primate, dog, cat, rabbit, or rodent. In some embodiments, the subject is a primate. In some embodiments, the subject is a human. In some embodiments, the human is from about 1 to about 90 years old, such as from about 10 to about 80 years old, from about 20 to about 70 years old, or from about 30 to about 60 years old. In some embodiments, the human is at least about or is about any of 21, 30, 40, 50, 60, 65, 70, 75, 80, or 85 years old. In some embodiments, the human is a child. In some embodiments, the human is less than about or about any of 21, 18, 15, 10, 5, 4, 3, 2, or 1 years old. In some embodiments, the subject is a female. In some embodiments, the subject is a male.

### Kits/Article of Manufacture

Disclosed herein, in certain embodiments, are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

### EXEMPLARY EMBODIMENTS

The present disclosure is further described by the following embodiments. The features of each of the embodiments are combinable with any of the other embodiments where appropriate and practical.

Embodiment P1 is a method for treating a cancer associated with RET kinase activity in a subject in need thereof, comprising administering to the subject an effective amount of a compound of Formula (I), or a pharmaceutically acceptable salt thereof, wherein the cancer has developed resistance following prior therapy and/or wherein the subject has a solvent front mutation in the RET kinase, and wherein the compound of Formula (I) has the following structure:

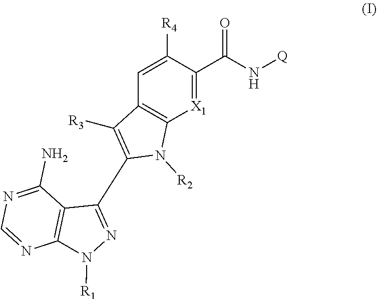

wherein:
X₁ is N or CH;
R₁ is C₁-C₆ alkyl or C₃-C₆ cycloalkyl, each of which is optionally substituted by 1-5 substituents independently selected from halo, hydroxy, and —O(C₁-C₆ alkyl);
R₂ is H or C₁-C₆ alkyl;
R₃ is halo or C₁-C₆ alkyl;
R₄ is H or halo; and
Q is C₁-C₆ alkyl or C₃-C₆ cycloalkyl, each of which is optionally substituted by 1-5 substituents independently selected from hydroxy, —O(C₁-C₆ alkyl), and halo.

Embodiment P2 is the method of embodiment P1, wherein:
X₁ is CH;
R₁ is C₃-C₆ alkyl optionally substituted by 1-3 substituents independently selected from halo, hydroxy, and —O(C₁-C₆ alkyl);
R₂ is H;
R₃ is halo;
R₄ is H; and
Q is C₁-C₃ alkyl or C₃-C₆ cycloalkyl, each of which is optionally substituted by 1-3 substituents independently selected from hydroxy and —O(C₁-C₃ alkyl).

Embodiment P3 is the method of embodiment 2, wherein:
R₁ is isopropyl or tert-butyl;
R₃ is Cl; and
Q is —CH₃, —CH₂CH₂OH, —(CH₂)₃O CH(CH₃)₂, or cyclopropyl.

Embodiment P4 is the method of any one of embodiments P1-P3, wherein the compound of Formula (I) is:

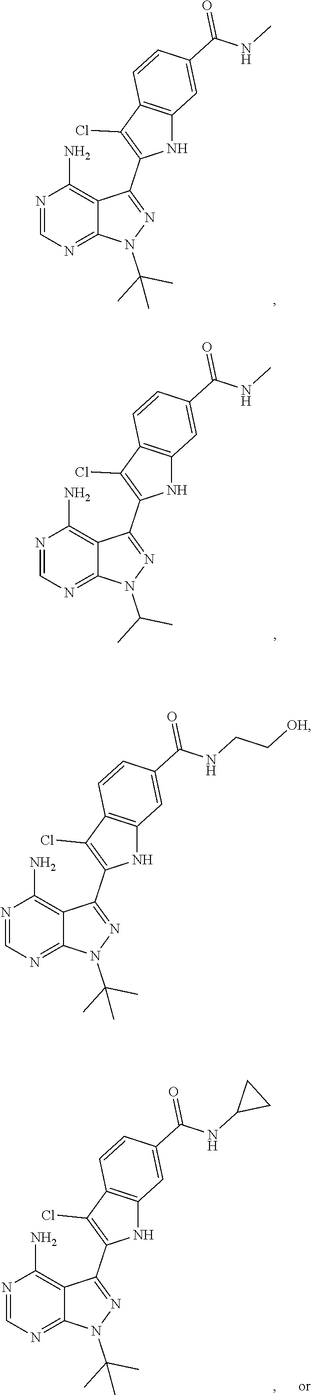 [SMILES: CNC(=O)c1ccc2c(Cl)c(-c3nn(C(C)(C)C)c4ncnc(N)c34)[nH]c2c1 ; CNC(=O)c1ccc2c(Cl)c(-c3nn(C(C)C)c4ncnc(N)c34)[nH]c2c1 ; CC(C)(C)n1nc(-c2[nH]c3cc(C(=O)NCCO)ccc3c2Cl)c2c(N)ncnc21 ; CC(C)(C)n1nc(-c2[nH]c3cc(C(=O)NC4CC4)ccc3c2Cl)c2c(N)ncnc21]

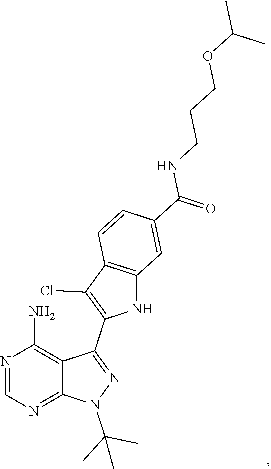 [SMILES: CC(C)OCCCNC(=O)c1ccc2c(Cl)c(-c3nn(C(C)(C)C)c4ncnc(N)c34)[nH]c2c1]

or a pharmaceutically acceptable salt thereof.

Embodiment P5 is the method of any one of embodiments P1-P4, wherein the compound of Formula (I) is:

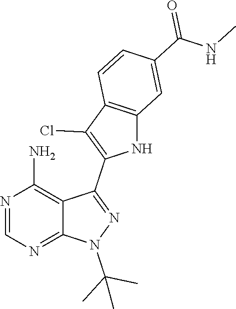 [SMILES: CNC(=O)c1ccc2c(Cl)c(-c3nn(C(C)(C)C)c4ncnc(N)c34)[nH]c2c1]

or a pharmaceutically acceptable salt thereof.

Embodiment P6 is the method of any one of embodiments P1-P4, wherein the compound of Formula (I) is:

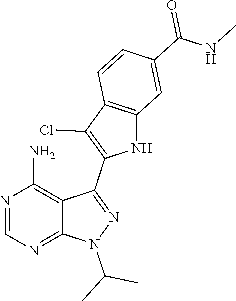 [SMILES: CNC(=O)c1ccc2c(Cl)c(-c3nn(C(C)C)c4ncnc(N)c34)[nH]c2c1]

or a pharmaceutically acceptable salt thereof.

Embodiment P7 is the method of any one of embodiments P1-P4, wherein the compound of Formula (I) is:

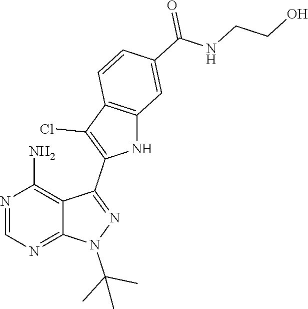 [SMILES: CC(C)(C)n1nc(-c2[nH]c3cc(C(=O)NCCO)ccc3c2Cl)c2c(N)ncnc21]

or a pharmaceutically acceptable salt thereof.

Embodiment P8 is the method of any one of embodiments P1-P4, wherein the compound of Formula (I) is:

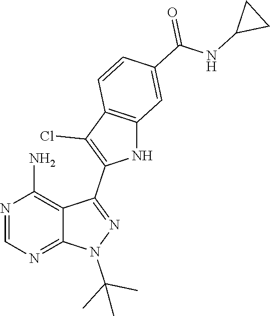 [SMILES: CC(C)(C)n1nc(-c2[nH]c3cc(C(=O)NC4CC4)ccc3c2Cl)c2c(N)ncnc21]

or a pharmaceutically acceptable salt thereof.

Embodiment P9 is the method of any one of embodiments P1-P4, wherein the compound of Formula (I) is:

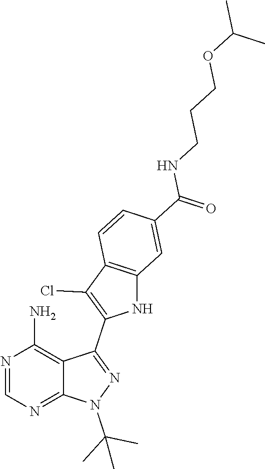 [SMILES: CC(C)OCCCNC(=O)c1ccc2c(Cl)c(-c3nn(C(C)(C)C)c4ncnc(N)c34)[nH]c2c1]

or a pharmaceutically acceptable salt thereof.

Embodiment P10 is the method of any one of embodiments P1-P9, wherein the subject previously responded to the prior therapy.

Embodiment P11 is the method of any one of embodiments P1-P10, wherein the subject no longer responds to the prior therapy.

Embodiment P12 is the method of any one of embodiments P1-P11, wherein the prior therapy is a kinase inhibitor, immunotherapy, chemotherapy, surgery, or radiation.

Embodiment P13 is the method of embodiment P12, wherein the prior therapy is a kinase inhibitor.

Embodiment P14 is the method of embodiment P13, wherein the kinase inhibitor is a selective RET kinase inhibitor or a multikinase inhibitor.

Embodiment P15 is the method of embodiment P14, wherein the selective RET kinase inhibitor is selpercatinib or pralsetinib.

Embodiment P16 is the method of embodiment P14, wherein the multikinase inhibitor is nintedanib, vandetanib, cabozantinib, lenvatinib, RXDX-105, sunitinib, sorafenib, alectinib, ponatinib, or regorafenib.

Embodiment P17 is the method of any one of embodiments P1-P16, wherein the cancer is a malignant neoplasm, a malignant tumor, or a solid tumor.

Embodiment P18 is the method of any one of embodiments P1-P17, wherein the cancer is leukemia, lung cancer, colon cancer, breast cancer, ovarian cancer, prostate cancer, liver cancer, pancreatic cancer, brain cancer, skin cancer, thyroid cancer, salivary gland cancer, endocrine cancer, urothelial cancer, uterine cancer, fallopian tube cancer, gastrointestinal cancer, or esophageal cancer.

Embodiment P19 is the method of embodiment P18, wherein the cancer is medullary thyroid cancer, non-small cell lung cancer, lung carcinosarcoma, lung adenocarcinoma, atypical lung carcinoid, multiple endocrine neoplasia type 2, ovarian epithelial carcinoma, uterine carcinosarcoma, fallopian tube adenocarcinoma, chronic myelomonocytic leukemia (CMML), melanoma, basal cell carcinoma, Merkel cell carcinoma, salivary gland adenocarcinoma, papillary thyroid carcinoma (PTC), anaplastic thyroid carcinoma, meningioma, esophageal adenocarcinoma, gastric adenocarcinoma, ureter urothelial carcinoma, duodenal adenocarcinoma, or colorectal adenocarcinoma.

Embodiment P20 is the method of any one of embodiments P1-P19, wherein the resistance of the cancer is due to a solvent front mutation in the RET kinase.

Embodiment P21 is the method of any one of embodiments P1-P20, wherein the solvent front mutation is at G810 in the amino acid sequence of the RET kinase.

Embodiment P22 is the method of any one of embodiments P1-P21, wherein the solvent front mutation is G810A, G810C, G810R, G810V, or G810S.

Embodiment P23 is the method of any one of embodiments P1-P22, wherein the RET kinase further comprises a RET fusion translocation and/or a mutation at the RET gatekeeper residue V804.

Embodiment P24 is the method of embodiment P23, wherein the RET fusion translocation is KIF5B-RET or CCDC6-RET.

Embodiment P25 is the method of embodiment P23 or P24, wherein the RET gatekeeper residue V804 is RET^{V804M}.

Embodiment P26 is the method of any one of embodiments P1-P25, wherein the solvent front mutation, the RET fusion translocation, and/or the mutation at the RET gatekeeper residue V804 in the RET kinase is identified by a detection method comprising evaluating circulating tumor (cell-free) DNA and/or evaluating a tissue biopsy.

Embodiment P27 is the method of embodiment P26, wherein the detection method comprises sequencing.

Embodiment P28 is the pharmaceutical composition for treating a subject with a cancer associated with RET kinase activity and wherein the cancer has developed resistance following prior therapy and/or wherein the subject has a solvent front mutation in the RET kinase, comprising a therapeutically effective amount of a compound of Formula (I):

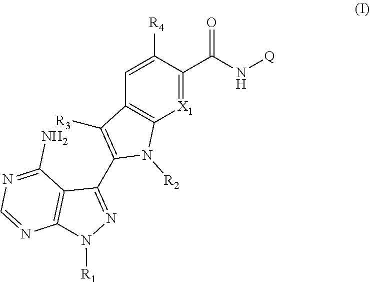

or a pharmaceutically acceptable salt thereof, wherein:
X₁ is N or CH;
R₁ is C₁-C₆ alkyl or C₃-C₆ cycloalkyl, each of which is optionally substituted by 1-5 substituents independently selected from halo, hydroxy, and —O(C₁-C₆ alkyl);
R₂ is H or C₁-C₆ alkyl;
R₃ is halo or C₁-C₆ alkyl;
R₄ is H or halo; and
Q is C₁-C₆ alkyl or C₃-C₆ cycloalkyl, each of which is optionally substituted by 1-5 substituents independently selected from hydroxy, —O(C₁-C₆ alkyl), and halo.

Embodiment P29 is the pharmaceutical composition of embodiment P28, wherein:
X₁ is CH;
R₁ is C₃-C₆ alkyl optionally substituted by 1-3 substituents independently selected from halo, hydroxy, and —O(C₁-C₆ alkyl);
R₂ is H;
R₃ is halo;
R₄ is H; and
Q is C₁-C₃ alkyl or C₃-C₆ cycloalkyl, each of which is optionally substituted by 1-3 substituents independently selected from hydroxy and —O(C₁-C₃ alkyl).

Embodiment P30 is the pharmaceutical composition of embodiment P29, wherein:
R₁ is isopropyl or tert-butyl;
R₃ is Cl; and
Q is —CH₃, —CH₂CH₂OH, —(CH₂)₃O CH(CH₃)₂, or cyclopropyl.

Embodiment P31 is the pharmaceutical composition of any one of embodiments P28-P30, wherein the compound of Formula (I) is:

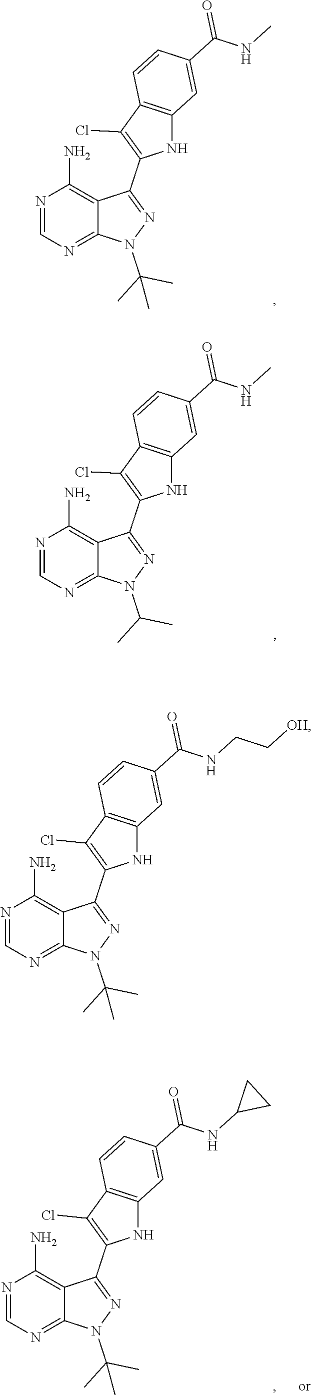 [SMILES: CNC(=O)c1ccc2c(Cl)c(-c3nn(C(C)(C)C)c4ncnc(N)c34)[nH]c2c1 ; CNC(=O)c1ccc2c(Cl)c(-c3nn(C(C)C)c4ncnc(N)c34)[nH]c2c1 ; CC(C)(C)n1nc(-c2[nH]c3cc(C(=O)NCCO)ccc3c2Cl)c2c(N)ncnc21 ; CC(C)(C)n1nc(-c2[nH]c3cc(C(=O)NC4CC4)ccc3c2Cl)c2c(N)ncnc21]

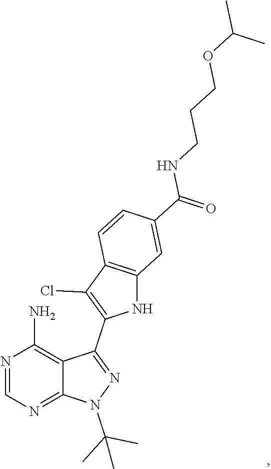 [SMILES: CC(C)OCCCNC(=O)c1ccc2c(Cl)c(-c3nn(C(C)(C)C)c4ncnc(N)c34)[nH]c2c1]

or a pharmaceutically acceptable salt thereof.

Embodiment P32 is the pharmaceutical composition of any one of embodiments P28-P31, wherein the compound of Formula (I) is:

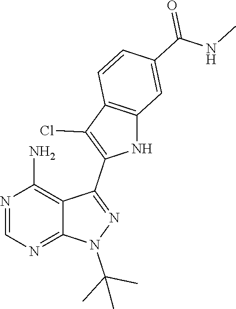 [SMILES: CNC(=O)c1ccc2c(Cl)c(-c3nn(C(C)(C)C)c4ncnc(N)c34)[nH]c2c1]

or a pharmaceutically acceptable salt thereof.

Embodiment P33 is the pharmaceutical composition of any one of embodiments P28-P31, wherein the compound of Formula (I) is:

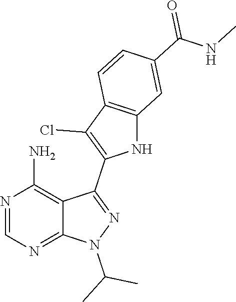 [SMILES: CNC(=O)c1ccc2c(Cl)c(-c3nn(C(C)C)c4ncnc(N)c34)[nH]c2c1]

or a pharmaceutically acceptable salt thereof.

Embodiment P34 is the pharmaceutical composition of any one of embodiments P28-P31, wherein the compound of Formula (I) is:

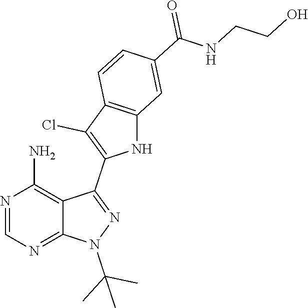 [SMILES: CC(C)(C)n1nc(-c2[nH]c3cc(C(=O)NCCO)ccc3c2Cl)c2c(N)ncnc21]

or a pharmaceutically acceptable salt thereof.

Embodiment P35 is the pharmaceutical composition of any one of embodiments P28-P31, wherein the compound of Formula (I) is:

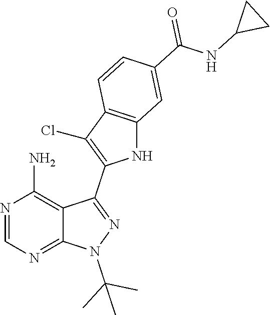 [SMILES: CC(C)(C)n1nc(-c2[nH]c3cc(C(=O)NC4CC4)ccc3c2Cl)c2c(N)ncnc21]

or a pharmaceutically acceptable salt thereof.

Embodiment P36 is the pharmaceutical composition of any one of embodiments P28-P31, wherein the compound of Formula (I) is:

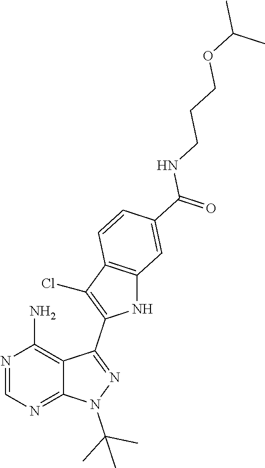 [SMILES: CC(C)OCCCNC(=O)c1ccc2c(Cl)c(-c3nn(C(C)(C)C)c4ncnc(N)c34)[nH]c2c1]

or a pharmaceutically acceptable salt thereof.

Embodiment P37 is the pharmaceutical composition of any one of embodiments P28-P36, wherein the subject previously responded to the prior therapy.

Embodiment P38 is the pharmaceutical composition of any one of embodiments P28-P37, wherein the subject no longer responds to the prior therapy.

Embodiment P39 is the pharmaceutical composition of any one of embodiments P28-P38, wherein the prior therapy is a kinase inhibitor, immunotherapy, chemotherapy, surgery, or radiation.

Embodiment P40 is the pharmaceutical composition of embodiment P39, wherein the prior therapy is a kinase inhibitor.

Embodiment P41 is the pharmaceutical composition of embodiment P40, wherein the kinase inhibitor is a selective RET kinase inhibitor or a multikinase inhibitor.

Embodiment P42 is the pharmaceutical composition of embodiment P41, wherein the selective RET kinase inhibitor is selpercatinib or pralsetinib.

Embodiment P43 is the pharmaceutical composition of embodiment P41, wherein the multikinase inhibitor is nintedanib, vandetanib, cabozantinib, lenvatinib, RXDX-105, sunitinib, sorafenib, alectinib, ponatinib, or regorafenib.

Embodiment P44 is the pharmaceutical composition of any one of embodiments P28-P43, wherein the cancer is a malignant neoplasm, a malignant tumor, or a solid tumor.

Embodiment P45 is the pharmaceutical composition of any one of embodiments P28-P44, wherein the cancer is leukemia, lung cancer, colon cancer, breast cancer, ovarian cancer, prostate cancer, liver cancer, pancreatic cancer, brain cancer, skin cancer, thyroid cancer, salivary gland cancer, endocrine cancer, urothelial cancer, uterine cancer, fallopian tube cancer, gastrointestinal cancer, or esophageal cancer.

Embodiment P46 is the pharmaceutical composition of embodiment P45, wherein the cancer is medullary thyroid cancer, non-small cell lung cancer, lung carcinosarcoma, lung adenocarcinoma, atypical lung carcinoid, multiple endocrine neoplasia type 2, ovarian epithelial carcinoma, uterine carcinosarcoma, fallopian tube adenocarcinoma, chronic myelomonocytic leukemia (CMML), melanoma, basal cell carcinoma, Merkel cell carcinoma, salivary gland adenocarcinoma, papillary thyroid carcinoma (PTC), anaplastic thyroid carcinoma, meningioma, esophageal adenocarcinoma, gastric adenocarcinoma, ureter urothelial carcinoma, duodenal adenocarcinoma, or colorectal adenocarcinoma.

Embodiment P47 is the pharmaceutical composition of any one of embodiments P28-P46, wherein the resistance of the cancer is due to a solvent front mutation in the RET kinase.

Embodiment P48 is the pharmaceutical composition of any one of embodiments P28-P47, wherein the solvent front mutation is at G810 in the amino acid sequence of the RET kinase.

Embodiment P49 is the pharmaceutical composition of any one of embodiments P28-P48, wherein the solvent front mutation is G810A, G810C, G810R, G810V, or G810S.

Embodiment P50 is the pharmaceutical composition of any one of embodiments P28-P49, wherein the RET kinase further comprises a RET fusion translocation and/or a mutation at the RET gatekeeper residue V804.

Embodiment 51 is the pharmaceutical composition of embodiment P50, wherein the RET fusion translocation is KIF5B-RET or CCDC6-RET.

Embodiment P52 is the pharmaceutical composition of embodiment P50 or P51, wherein the RET gatekeeper residue V804 is RET^{V804M}.

Embodiment P53 is the pharmaceutical composition of any one of embodiments P28-P52, wherein the solvent front mutation, the RET fusion translocation, and/or the mutation at the RET gatekeeper residue V804 in the RET kinase is identified by a detection method comprising evaluating circulating tumor DNA and/or evaluating a tissue biopsy.

Embodiment P54 is the pharmaceutical composition of embodiment P53, wherein the detection method comprises sequencing.

Embodiment P55 is a method of inhibiting a mutant RET kinase, comprising contacting the mutant RET kinase with an effective amount of a compound of Formula (I):

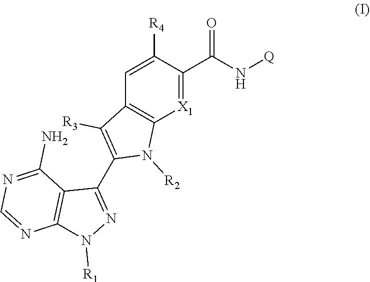

or a pharmaceutically acceptable salt thereof, wherein:
X₁ is N or CH;
R₁ is C₁-C₆ alkyl or C₃-C₆ cycloalkyl, each of which is optionally substituted by 1-5 substituents independently selected from halo, hydroxy, and —O(C₁-C₆ alkyl);
R₂ is H or C₁-C₆ alkyl;
R₃ is halo or C₁-C₆ alkyl;
R₄ is H or halo; and
Q is C₁-C₆ alkyl or C₃-C₆ cycloalkyl, each of which is optionally substituted by 1-5 substituents independently selected from hydroxy, —O(C₁-C₆ alkyl), and halo.

Embodiment P56 is the method of embodiment P55, wherein:
X₁ is CH;
R₁ is C₃-C₆ alkyl optionally substituted by 1-3 substituents independently selected from halo, hydroxy, and —O(C₁-C₆ alkyl);
R₂ is H;
R₃ is halo;
R₄ is H; and
Q is C₁-C₃ alkyl or C₃-C₆ cycloalkyl, each of which is optionally substituted by 1-3 substituents independently selected from hydroxy and —O(C₁-C₃ alkyl).

Embodiment P57 is the method of embodiment P56, wherein:
R₁ is isopropyl or tert-butyl;
R₃ is Cl; and
Q is —CH₃, —CH₂CH₂OH, —(CH₂)₃O CH(CH₃)₂, or cyclopropyl.

Embodiment P58 is the method of any one of embodiments P55-P57, wherein the compound of Formula (I) is:

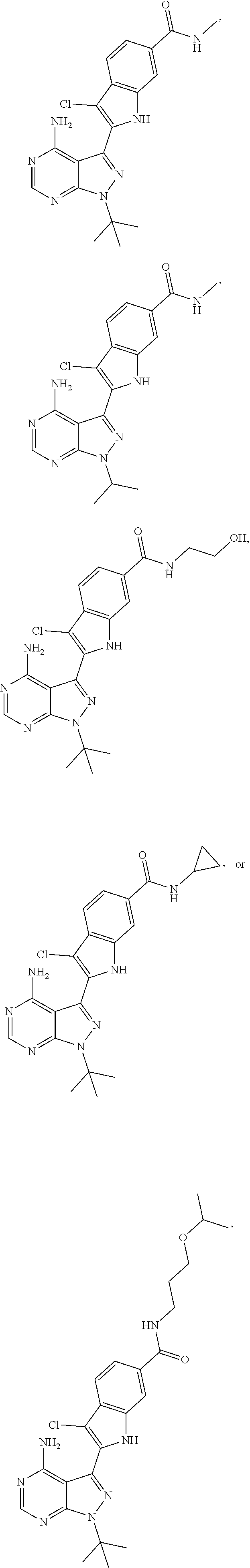 [SMILES: CNC(=O)c1ccc2c(Cl)c(-c3nn(C(C)(C)C)c4ncnc(N)c34)[nH]c2c1 ; CNC(=O)c1ccc2c(Cl)c(-c3nn(C(C)C)c4ncnc(N)c34)[nH]c2c1 ; CC(C)(C)n1nc(-c2[nH]c3cc(C(=O)NCCO)ccc3c2Cl)c2c(N)ncnc21 ; CC(C)(C)n1nc(-c2[nH]c3cc(C(=O)NC4CC4)ccc3c2Cl)c2c(N)ncnc21 ; CC(C)OCCCNC(=O)c1ccc2c(Cl)c(-c3nn(C(C)(C)C)c4ncnc(N)c34)[nH]c2c1]

or a pharmaceutically acceptable salt thereof.

Embodiment P59 is the method of any one of embodiments P55-P58, wherein the compound of Formula (I) is:

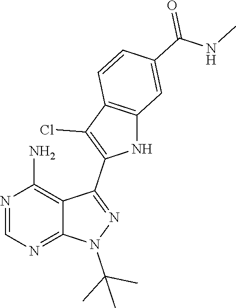 [SMILES: CNC(=O)c1ccc2c(Cl)c(-c3nn(C(C)(C)C)c4ncnc(N)c34)[nH]c2c1]

or a pharmaceutically acceptable salt thereof.

Embodiment P60 is the method of any one of embodiments P55-P58, wherein the compound of Formula (I) is:

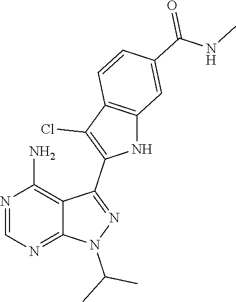 [SMILES: CNC(=O)c1ccc2c(Cl)c(-c3nn(C(C)C)c4ncnc(N)c34)[nH]c2c1]

or a pharmaceutically acceptable salt thereof.

Embodiment P61 is the method of any one of embodiments P55-P58, wherein the compound of Formula (I) is:

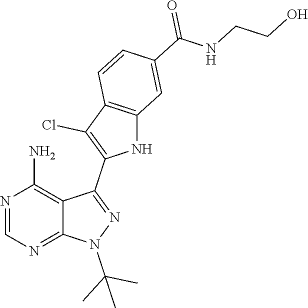 [SMILES: CC(C)(C)n1nc(-c2[nH]c3cc(C(=O)NCCO)ccc3c2Cl)c2c(N)ncnc21]

or a pharmaceutically acceptable salt thereof.

Embodiment P62 is the method of any one of embodiments P55-P58, wherein the compound of Formula (I) is:

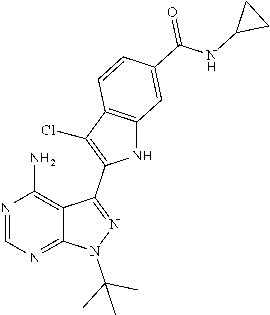 [SMILES: CC(C)(C)n1nc(-c2[nH]c3cc(C(=O)NC4CC4)ccc3c2Cl)c2c(N)ncnc21]

or a pharmaceutically acceptable salt thereof.

Embodiment P63 is the method of any one of embodiments P55-P58, wherein the compound of Formula (I) is:

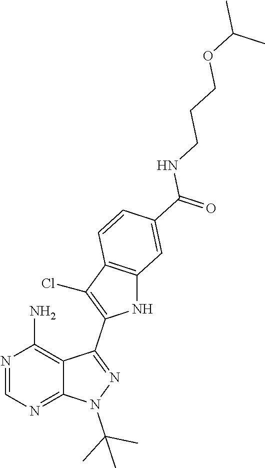 [SMILES: CC(C)OCCCNC(=O)c1ccc2c(Cl)c(-c3nn(C(C)(C)C)c4ncnc(N)c34)[nH]c2c1]

or a pharmaceutically acceptable salt thereof.

Embodiment P64 is the method of any one of embodiments P55-P63, wherein the method is an in vitro method.

Embodiment P65 is the method of any one of embodiments P55-P63, wherein the method is an in vivo method.

Embodiment P66 is the method of any one of embodiments P55-P65, wherein the mutant RET kinase is due to resistance developed from a prior therapy.

Embodiment P67 is the method of embodiment P66, wherein the prior therapy is a kinase inhibitor, immunotherapy, chemotherapy, surgery, or radiation.

Embodiment P68 is the method of embodiment P67, wherein the prior therapy is a kinase inhibitor.

Embodiment P69 is the method of embodiment P68, wherein the kinase inhibitor is a selective RET kinase inhibitor or a multikinase inhibitor.

Embodiment P70 is the method of embodiment P69, wherein the selective RET kinase inhibitor is selpercatinib or pralseltinib.

Embodiment P71 is the method of embodiment P69, wherein the multikinase inhibitor is nintedanib, vandetanib, cabozantinib, lenvatinib, RXDX-105, sunitinib, sorafenib, alectinib, ponatinib, or regorafenib.

Embodiment P72 is the method of any one of embodiments P55-P71, wherein the mutant RET kinase comprises a solvent front mutation.

Embodiment P73. The method of embodiment P72, wherein the solvent front mutation is at G810 in the amino acid sequence of the RET kinase.

Embodiment P74 is the method of embodiment P73, wherein the solvent front mutation is G810A, G810C, G810R, G810V, or G810S.

Embodiment P75 is the method of any one of embodiments P55-P74, wherein the RET kinase further comprises a RET fusion translocation and/or a mutation at the RET gatekeeper residue V804.

Embodiment 76 is the method of embodiment P75, wherein the RET fusion translocation is KIF5B-RET or CCDC6-RET.

Embodiment P77 is the method of embodiment P75 or P76, wherein the RET gatekeeper residue V804 is RET^{V804M}.

### EXAMPLES

These examples are provided for illustrative purposes only and not to limit the scope of the claims provided herein.

The following abbreviations may be relevant for the application.

### Abbreviations

ATP: adenosine triphosphate
CSK: C-terminal Src kinase
DMSO: dimethylsulfoxide
DTT: dithiothreitol
FBS: fetal bovine serum
h: hour
MSA: mobility shift assay
PBS: phosphate buffered saline
TKI: tyrosine kinase inhibitor
WT: wild type

### Example 1. In Vitro RET Kinase Enzyme Assays

### Materials.

Wild-type human RET kinase (RET WT; cytoplasmic domain, amino acids 658-1114 of accession number NP_066124.1) was expressed as an N-terminal GST-fusion protein (79 kDa) in a baculovirus expression system and purified using glutathione sepharose chromatography.

Human RET kinase G810 mutants (cytoplasmic domain, amino acids 658-1114 of accession number NP_066124.1, mutant [G810C], [G810R] or [G810S]) were expressed as N-terminal DYKDDDK-tagged (SEQ ID NO: 3) biotinylated protein (56 kDa) in a baculovirus expression system and activated with ATP. The protein was purified using DYKDDDK tag (SEQ ID NO: 3) antibody agarose and activated with ATP. The activated protein was purified using gel filtration.

2× kinase solutions (containing 2 mM RET kinase) were prepared using an assay buffer containing 20 mM HEPES pH 7.5, 0.01% Triton X-100, and 1 mM DTT.

A 4× Substrate/ATP/Metal solution was prepared using a kit buffer containing 20 mM HEPES pH 7.5, Triton X-100, and 5 mM DTT such that the final 1× Substrate/ATP/Metal solution would be 1/1000/5000 μM. The substrate t was the synthetic peptide CSKtide peptide conjugated to a fluorophore, and the metal was MgCl₂.

The compound 2-(4-Amino-1-tert-butyl-pyrazolo[3,4-d]pyrimidin-3-yl)-3-chloro-N-methyl-1H-indole-6-carboxamide (referred to herein as “Compound A”) was prepared according to Example 4 in PCT Publication No. WO 2017/178844. The compounds selpercatinib, pralsetinib, and staurosporine, all of which are TKIs, were obtained from commercial sources. Solutions of Compound A, selpercatinib, pralsetinib, and staurosporine were each prepared in DMSO at concentrations 100-fold higher than the reaction condition and further diluted 25-fold with assay buffer for a 4 compound solution. Final 1× concentrations of Compound A, selpercatinib, and pralsetinib were 10 μM-0.3 nM; final 1× concentrations of staurosporine were 0.1 μM-0.003 nM (RET WT) or 10 μM-0.3 nM (RET G810C, G810R, G810S).

### Procedure.

5 μL of 4× compound solution, 5 μL of 4× Substrate/ATP/Metal solution, and 10 L of 2× kinase solution were mixed in each well of a 384-well plate. After incubation for 1 h at room temperature, 70 μL of Termination Buffer (QuickScout Screening Assist MSA; Carna Biosciences) was added to each well. Tyrosine kinase activities were measured using an off-chip mobility shift assay (MSA) by applying the reaction mixture on a LabChip™ system (Perkin Elmer) which quantified the product (P) and substrate (S) peptide peaks. The kinase reaction was evaluated based on the product ratio (P/(P+S)) calculated from peak intensities.

Percent inhibition of each test solution was calculated by setting the readout value of reaction control (complete reaction mixture for each kinase without TKI) as 0% inhibition and the readout value of background (reaction mixture without enzyme) as 100% inhibition. IC₅₀ values were calculated from concentration vs. % inhibition curves by fitting to a four-parameter logistic curve.

### Results.

Activity data (IC₅₀) for the tested compounds against wild type RET kinase (RET WT) and mutant RET kinases are shown in Table 3 below.

TABLE 3
Inhibition data of RET kinases.

| | IC₅₀ (M) | | | |
|---|---|---|---|---|
| Kinase | Compound A | Selpercatinib | Pralsetinib | Staurosporine |
| RET WT | 2.53E−08 | 8.84E−09 | 1.50E−08 | 1.70E−08 |
| RET G810C | 4.08E−08 | 6.72E−07 | 2.71E−07 | 2.96E−07 |
| RET G810R | 8.09E−08 | 9.77E−07 | 1.32E−06 | 1.07E−06 |
| RET G810S | 3.36E−08 | 1.25E−07 | 5.98E−08 | 2.63E−07 |

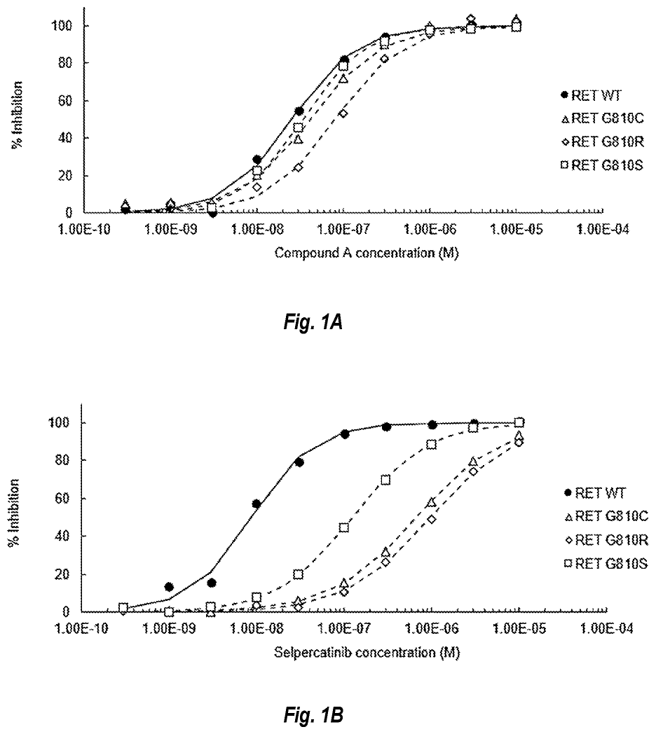

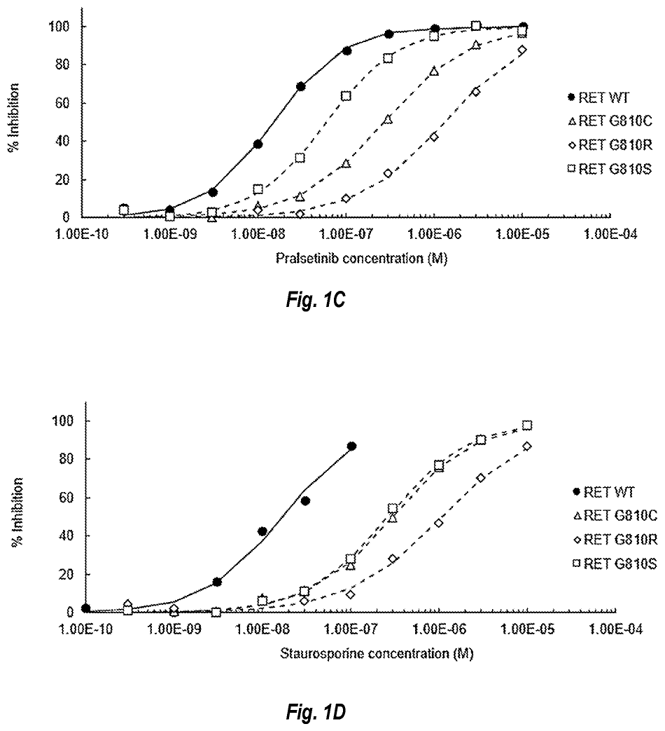

Inhibition curves of each of the tested compounds against RET WT and mutant RET kinases are shown in FIGS. 1A-D.

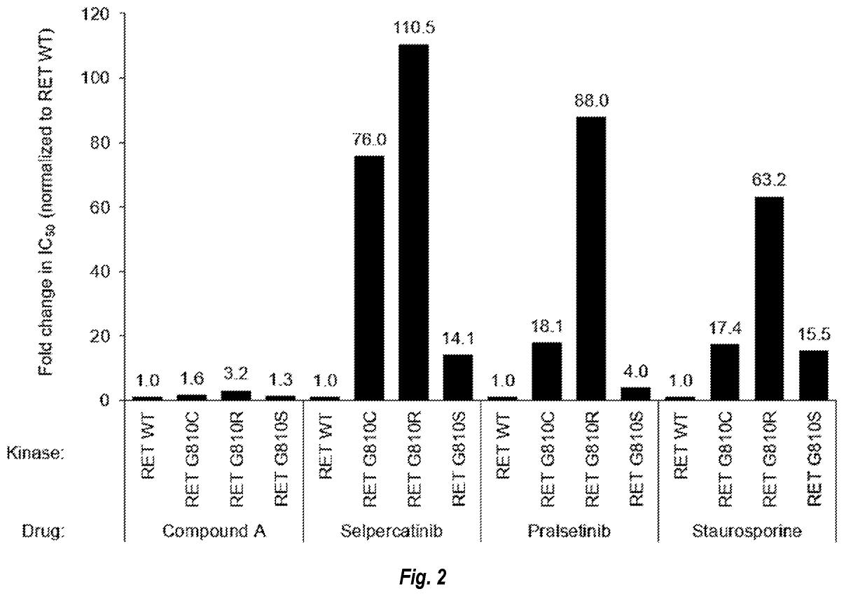

Fold changes in IC₅₀ values for each of the compounds tested against mutant RET kinases, normalized to RET WT, are shown in FIG. 2.

Overall, the data demonstrate that Compound A exhibits consistent efficacy against both RET WT and the tested G810 mutant RET kinases. In contrast, the two clinically approved TKIs, selpercatinib and pralsetinib, and the widely used reagent, staurosporine, all showed a significant decrease in inhibitory activity toward the mutant RET kinases, with an approximately 10-100 fold increase in IC₅₀ values.

### Example 2. In Vitro Cell Viability Assays

### Materials.

The following reagents were obtained commerically from the indicated suppliers: RPMI 1640 media (Hyclone), FBS (Gibco), PBS (Solarbio), DMSO (Sigma), and CellTiter-Glo® luminescent cell viability assay (Vazyme). The CellTiter-Glo® Luminescent Cell Viability Assay is a homogeneous method to determine the number of viable cells in culture based on quantitation of the amount of ATP present, which is directly proportional to the number of viable, metabolically active cells. The homogeneous “add-mix-measure” format results in cell lysis and generation of a luminescent signal proportional to the amount of ATP present.

Ba/F3 mouse pro-B cell lines transduced KIF5B-RET fusion gene were used in this study. The KIF5B-RET fusion gene, a result of gene rearrangement, is expressed in a subset of cancers such as non-small cell lung cancer, and consists of the 5′ portion of the KIF5B gene fused in-frame to the 3′ portion of the RET gene (encoding the RET kinase domain). Ba/F3 cells stably expressing KIF5B-RET fusion proteins, in which the RET domain is wildtype or harbors mutations at G810 (G810R, G810C, G810S), were used in these experiments. Ba/F3 KIF5B-RET (wild type RET kinase, cell line no. KC-1041), Ba/F3 KIF5B-RET-G810R (G810R mutant RET kinase, cell line no. KC-1623), Ba/F3 KIF5B-RET-G810C (G810C mutant RET kinase, cell line no. KC-1623), and Ba/F3 KIF5B-RET-G810S (G810S mutant RET kinase, cell line no. KC-1556) were cultured in RPMI 1640 media supplemented with 10% FBS.

Compound A, described in Example 1, was used in this study, as well as the two clinically approved TKIs pralsetinib and selpercatinib.

### Procedure (Cytotoxicity and IC₅₀).

Cells were harvested during the logarithmic growth period and counted using a blood counting chamber. The cell viability was over 90%, as determined by trypan blue assay. The cell concentration was adjusted with culture medium, and cell suspensions were added to two 96-well plates to give a final cell density of 3,000 cells per well. The plates were incubated in a humidified incubator at 37° C. with 5% CO₂.

Stock solutions of Compound A, pralsetinib, and selpercatinib were prepared in DMSO. Cells were incubated with 9 concentrations of each compound (3.16-fold serial dilution) ranging from 10 μM to 1 nM. The final concentration of DMSO in the culture medium was 0.1% [v/v]).

The plates were cultured for 3 days. On Day 3, CellTiter-Glo® Reagent was added to each well, and the contents of each well were mixed for 5 minutes on an orbital shaker to induce cell lysis. The cell plate was incubated at room temperature for 20 minutes to stabilize the luminescent signal, and luminescence was recorded using a Multi-mode Microplate Reader.

GraphPad Prism 7.0 was used to calculate IC₅₀ values. The graphical curves were fitted using a nonlinear regression model with a sigmoidal dose response.

Cell viability was determined according to equation (1):

Cell viability (%)=(Lum_{test cmpd}−Lum_{medium control})/(Lum_{vehicle control}-Lum_{medium control}) (equation 1)
Lum=luminescence
test cmpd=test compound (targeting RET kinase)

### Results.

IC₅₀ data for the tested compounds against Ba/F3 KIF5B-RET cell lines with wild type RET or mutant RET kinase domains are shown in Table 4 below.

TABLE 4
Summary of IC₅₀ Data.

| | IC₅₀ (nM) | | |
|---|---|---|---|
| Cell line | Compound A | pralsetinib | selpercatinib |
| Ba/F3 KIF5B-RET | 20.0 | 29.6 | 24.4 |
| Ba/F3 KIF5B-RET-G810R | 37.4 | 1300.1 | 986.4 |
| Ba/F3 KIF5B-RET-G810C | 42.4 | 551.2 | 1075.8 |
| Ba/F3 KIF5B-RET-G810S | 28.7 | 90.5 | 204.2 |

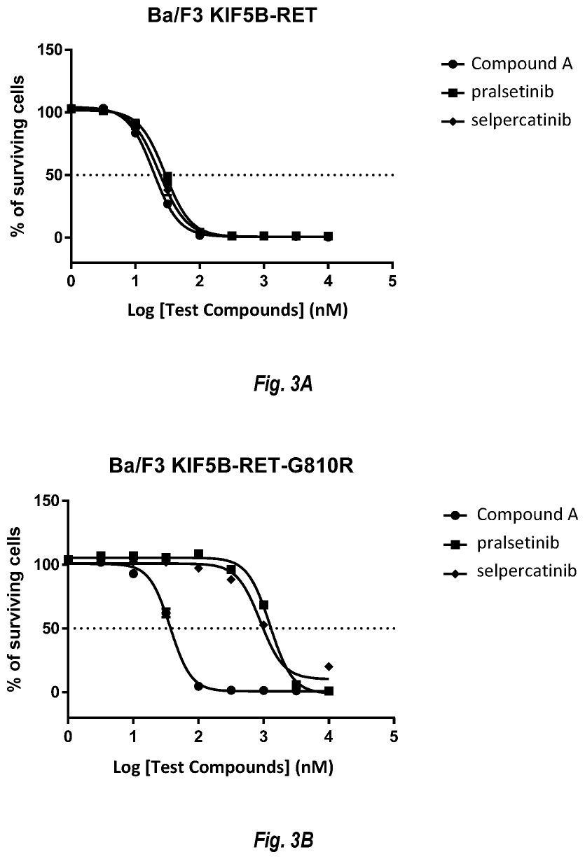

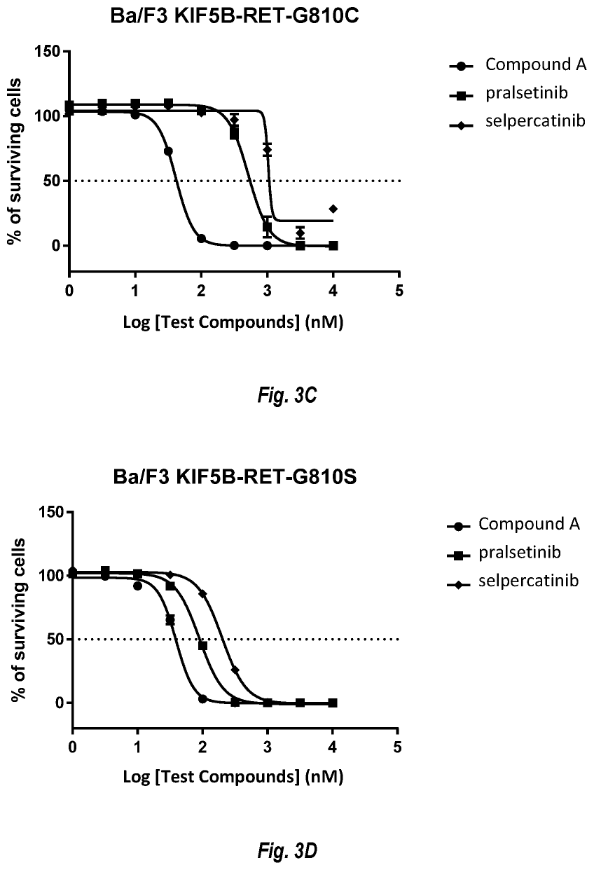

Cell viability curves for each of the tested compounds against Ba/F3 KIF5B-RET cell lines with wild type RET or mutant RET kinase domains are shown in FIGS. 3A-D.

The data demonstrate that Compound A is extremely potent against Ba/F3 cells expressing KIF5B-RET with a wild type RET domain as well as G810 RET mutants, as determined by IC₅₀ values that were largely unperturbed by the presence of the G810 mutations. In contrast, selpercatinib and pralsetinib showed an increase in IC₅₀ values against Ba/F3 cells expressing KIF5B-RET with a G810 RET mutant domain, indicating increased cell viability in the mutant RET kinase cell lines treated with the two approved compounds. Specifically, there was an 8-44-fold increase in selpercatinib IC₅₀ values and a 3-44-fold increase in pralsetinib IC₅₀ values against mutant RET kinase cell lines as compared to wild type RET kinase cell lines. These results are consistent with the results of the in vitro RET kinase enzyme assays of Example 1.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. The disclosures of all patent and scientific literature cited herein are expressly incorporated herein in their entirety by reference.

1. A method for treating a cancer associated with RET kinase activity in a subject in need thereof, comprising administering to the subject an effective amount of a compound of Formula (I), or a pharmaceutically acceptable salt thereof, wherein the cancer has developed resistance following prior therapy and/or wherein the subject has a solvent front mutation in the RET kinase, and wherein the compound of Formula (I) has the following structure: 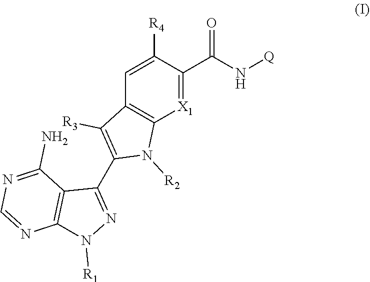
wherein:
X₁ is N or CH;
R₁ is C₁-C₆ alkyl or C₃-C₆ cycloalkyl, each of which is optionally substituted by 1-5 substituents independently selected from halo, hydroxy, and —O(C₁-C₆ alkyl);
R₂ is H or C₁-C₆ alkyl;
R₃ is halo or C₁-C₆ alkyl;
R₄ is H or halo; and
Q is C₁-C₆ alkyl or C₃-C₆ cycloalkyl, each of which is optionally substituted by 1-5 substituents independently selected from hydroxy, —O(C₁-C₆ alkyl), and halo.

2. The method of claim 1, wherein:
X₁ is CH;
R₁ is C₃-C₆ alkyl optionally substituted by 1-3 substituents independently selected from halo, hydroxy, and —O(C₁-C₆ alkyl);
R₂ is H;
R₃ is halo;
R₄ is H; and
Q is C₁-C₃ alkyl or C₃-C₆ cycloalkyl, each of which is optionally substituted by 1-3 substituents independently selected from hydroxy and —O(C₁-C₃ alkyl).

3. The method of claim 2, wherein:
R₁ is isopropyl or tert-butyl;
R₃ is Cl; and
Q is —CH₃, —CH₂CH₂OH, —(CH₂)₃O CH(CH₃)₂, or cyclopropyl.

4. The method of claim 1, wherein the compound of Formula (I) is: 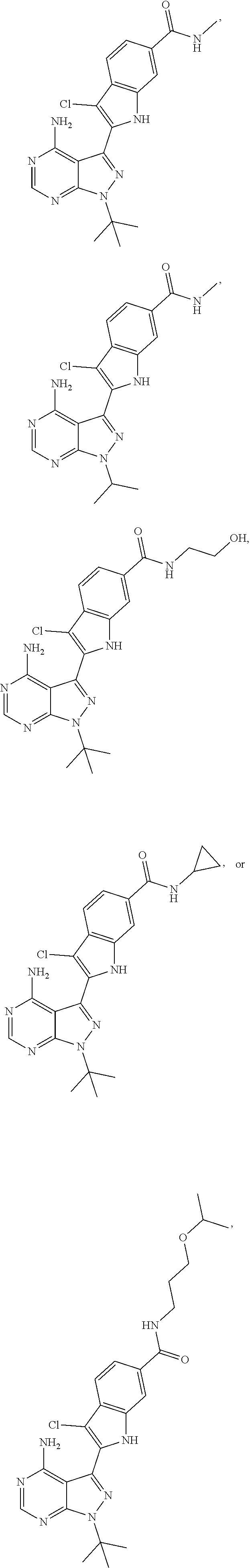 [SMILES: CNC(=O)c1ccc2c(Cl)c(-c3nn(C(C)(C)C)c4ncnc(N)c34)[nH]c2c1 ; CNC(=O)c1ccc2c(Cl)c(-c3nn(C(C)C)c4ncnc(N)c34)[nH]c2c1 ; CC(C)(C)n1nc(-c2[nH]c3cc(C(=O)NCCO)ccc3c2Cl)c2c(N)ncnc21 ; CC(C)(C)n1nc(-c2[nH]c3cc(C(=O)NC4CC4)ccc3c2Cl)c2c(N)ncnc21 ; CC(C)OCCCNC(=O)c1ccc2c(Cl)c(-c3nn(C(C)(C)C)c4ncnc(N)c34)[nH]c2c1]
or a pharmaceutically acceptable salt thereof.

5-10. (canceled)

11. The method of claim 1, wherein the subject no longer responds to the prior therapy, wherein the prior therapy is selected from a kinase inhibitor, immunotherapy, chemotherapy, surgery, and radiation.

12. (canceled)

13. The method of claim 11, wherein the prior therapy is a kinase inhibitor, and wherein the kinase inhibitor is a selective RET kinase inhibitor or a multikinase inhibitor.

14. (canceled)

15. The method of claim 13, wherein
(i) the selective RET kinase inhibitor is selpercatinib or pralsetinib; or
(ii) the multikinase inhibitor is nintedanib, vandetanib, cabozantinib, lenvatinib, RXDX-105, sunitinib, sorafenib, alectinib, ponatinib, or regorafenib.

16. (canceled)

17. (canceled)

18. The method of claim 1, wherein the cancer is selected from leukemia, lung cancer, colon cancer, breast cancer, ovarian cancer, prostate cancer, liver cancer, pancreatic cancer, brain cancer, skin cancer, thyroid cancer, salivary gland cancer, endocrine cancer, urothelial cancer, uterine cancer, fallopian tube cancer, gastrointestinal cancer, esophageal cancer, medullary thyroid cancer, non-small cell lung cancer, lung carcinosarcoma, lung adenocarcinoma, atypical lung carcinoid, multiple endocrine neoplasia type 2, ovarian epithelial carcinoma, uterine carcinosarcoma, fallopian tube adenocarcinoma, chronic myelomonocytic leukemia (CMML), melanoma, basal cell carcinoma, Merkel cell carcinoma, salivary gland adenocarcinoma, papillary thyroid carcinoma (PTC), anaplastic thyroid carcinoma, meningioma, esophageal adenocarcinoma, gastric adenocarcinoma, ureter urothelial carcinoma, duodenal adenocarcinoma, and colorectal adenocarcinoma.

19. (canceled)

20. The method of claim 1, wherein the resistance of the cancer is due to a solvent front mutation in the RET kinase.

21. The method of claim 1, wherein the solvent front mutation is at G810 in the amino acid sequence of the RET kinase, and wherein the solvent front mutation is selected from G810A, G810C, G810R, G810V, and G810S.

22. (canceled)

23. The method of claim 1, wherein the RET kinase further comprises a RET fusion translocation and/or a mutation at the RET gatekeeper residue V804, wherein the RET fusion translocation is KIF5B-RET or CCDC6-RET and/or the RET gatekeeper residue V804 is RET^{V804M}.

24. (canceled)

28. A method of inhibiting a mutant RET kinase, comprising contacting the mutant RET kinase with an effective amount of a compound of Formula (I): 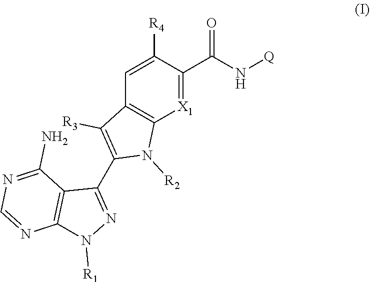
or a pharmaceutically acceptable salt thereof, wherein:
X₁ is N or CH;
R₁ is C₁-C₆ alkyl or C₃-C₆ cycloalkyl, each of which is optionally substituted by 1-5 substituents independently selected from halo, hydroxy, and —O(C₁-C₆ alkyl);
R₂ is H or C₁-C₆ alkyl;
R₃ is halo or C₁-C₆ alkyl;
R₄ is H or halo; and
Q is C₁-C₆ alkyl or C₃-C₆ cycloalkyl, each of which is optionally substituted by 1-5 substituents independently selected from hydroxy, —O(C₁-C₆ alkyl), and halo.

29. The method of claim 28, wherein:
X₁ is CH;
R₁ is C₃-C₆ alkyl optionally substituted by 1-3 substituents independently selected from halo, hydroxy, and —O(C₁-C₆ alkyl);
R₂ is H;
R₃ is halo;
R₄ is H; and
Q is C₁-C₃ alkyl or C₃-C₆ cycloalkyl, each of which is optionally substituted by 1-3 substituents independently selected from hydroxy and —O(C₁-C₃ alkyl).

30. The method of claim 29, wherein:
R₁ is isopropyl or tert-butyl;
R₃ is Cl; and
Q is —CH₃, —CH₂CH₂OH, —(CH₂)₃O CH(CH₃)₂, or cyclopropyl.

31. The method of claim 28, wherein the compound of Formula (I) is: 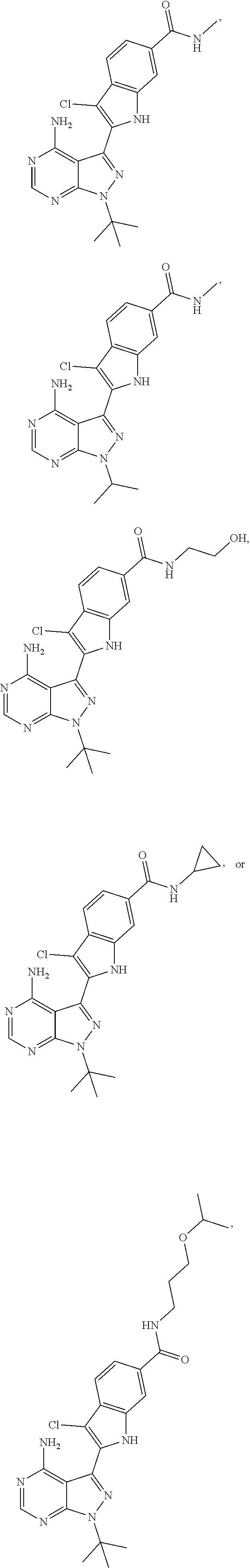 [SMILES: CNC(=O)c1ccc2c(Cl)c(-c3nn(C(C)(C)C)c4ncnc(N)c34)[nH]c2c1 ; CNC(=O)c1ccc2c(Cl)c(-c3nn(C(C)C)c4ncnc(N)c34)[nH]c2c1 ; CC(C)(C)n1nc(-c2[nH]c3cc(C(=O)NCCO)ccc3c2Cl)c2c(N)ncnc21 ; CC(C)(C)n1nc(-c2[nH]c3cc(C(=O)NC4CC4)ccc3c2Cl)c2c(N)ncnc21 ; CC(C)OCCCNC(=O)c1ccc2c(Cl)c(-c3nn(C(C)(C)C)c4ncnc(N)c34)[nH]c2c1]
or a pharmaceutically acceptable salt thereof.

32-36. (canceled)

37. (canceled)

38. The method of claim 28, wherein the method is an in vivo method.

39. The method of claim 28, wherein the mutant RET kinase is due to resistance developed from a prior therapy, wherein the prior therapy is selected from a kinase inhibitor, immunotherapy, chemotherapy, surgery, and radiation.

40-41. (canceled)

42. The method of claim 41, wherein the kinase inhibitor is a selective RET kinase inhibitor or a multikinase inhibitor, wherein the selective RET kinase inhibitor is selpercatinib or pralseltinib, and wherein the multikinase inhibitor is nintedanib, vandetanib, cabozantinib, lenvatinib, RXDX-105, sunitinib, sorafenib, alectinib, ponatinib, or regorafenib.

43-44. (canceled)

45. The method of claim 28, wherein the mutant RET kinase comprises a solvent front mutation, wherein the solvent front mutation is at G810 in the amino acid sequence of the RET kinase, and wherein the solvent front mutation is selected from G810A, G810C, G810R, G810V, and G810S.

46-47. (canceled)

48. The method of claim 28, wherein the RET kinase further comprises a RET fusion translocation and/or a mutation at the RET gatekeeper residue V804, wherein the RET fusion translocation is KIF5B-RET or CCDC6-RET and/or the RET gatekeeper residue V804 is RET^{V804M}.

49-50. (canceled)


SEQUENCE LISTING (3 sequences)

SEQ ID NO: 1 - protein, 457 aa, Homo sapiens
HCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 2 - protein, 1114 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 3 - protein, 7 aa, synthetic construct
DYKDDDK